SEQUENCE LISTING (27 sequences)

SEQ ID NO: 1 - DNA, 20 bp, Artificial Sequence
  Primer
catcgcgctgtctactcttg

SEQ ID NO: 2 - DNA, 20 bp, Artificial Sequence
  Primer
ttggctcgtcatagcggatc

SEQ ID NO: 3 - DNA, 122 bp, Feline coronavirus
catcgcgctgtctactcttgtacagaatggtaagcacgtgtaataggaggtacaagcaaccctattgcatattaggaagtttagatttgatttggcaatgctagatttagtaatttagagaa

SEQ ID NO: 4 - DNA, 166 bp, Feline coronavirus
tccgaggaattgctggtcatcgcgctgtctactcttgtacagaatggtaagcacgtgtaataggaggtacaagcaaccctattgcatattaggaagtttagatttgatttggcaatgctagatttagtaatttagagaagtttaaagatccgctatgacgagccaa

SEQ ID NO: 5 - protein, 16 aa, SARS coronavirus
ILPDPLKPTKRSFIED

SEQ ID NO: 6 - protein, 15 aa, Homo sapiens
ISPDQLADLYKFIKD

SEQ ID NO: 7 - protein, 11 aa, SARS coronavirus
NFSQILPDPLK

SEQ ID NO: 8 - protein, 14 aa, SARS coronavirus
FIEDLLFNKVTLAD

SEQ ID NO: 9 - DNA, 1694 bp, Felis cattus
attcaccatgtctattctcaagatccatgccagagagatcttcgactctcgtgggaatcccaccgttgaggtggacctctatacctccaaaggtctcttcagagccgctgtgcccagtggtgcctcaactggtatctacgaggccctcgagctccgggacaatgacaagacacgctacatgggcaaaggtgtctcaaaggctgttgagcacatcaataaaactattgcacctgccctgattagcaagaaactgagcgttgtggagcaggagaagattgacaaactgatgatcgagatggacgggacagaaaataaatctaaatttggtgcaaacgccatcctgggagtgtccctggcagtctgcaaggcgggggccgttgagaagggggtgcctctgtaccgccacattgccgacctggctggcaatgctgaagtcatcctgccggttccggctttcaatgtcatcaatggcggttctcacgccggcaacaagctggccatgcaagagttcatgatcctccccgttggtgccgccaacttcagggaagctatgcgcatcggtgcggaggtttaccacaacctgaagaacgtcatcaaggagaaatacggaaaagacgccaccaacgtgggggacgaaggcggctttgcccctaacatcctggagaacaaagaagctctggagctgctgaagaacgccatcgggaaagccggctacaccgacaaggtcgtcatcggcatggacgtggccgcttctgagttcttcaggtcggggaagtatgacctggacttcaagtctcccgatgaccccagcaggtacatcacaccggacgagctggccaacctgtacaagtccttcatcagggactacccagtggtgtccatcgaagatcccttcgaccaggatgactgggaagcttggcagaagttcactgccagcgccggcatccaggtggtgggggatgatctcaccgtgaccaacccgaagcggatttccaaggccgtgaacgagaggtcgtgcaactgcctcctgctcaaagtgaaccagatcggctctgtgaccgagtccctccaggcgtgcaagctggcccagtccaacgggtggggcgtcatggtgtcgcatcgctccggggagaccgaggataccttcatcgctgacctggtggtgggactttgcaccgggcagatcaagacaggtgcaccttgccgttctgagcgcctggccaagtacaaccagatcctcagaattgaagaggaactgggtagcaaggccaagtttgccggcagaagcttcagaaaccccctcgccaagtaagctccgggcgaggcaccccgagctctgcggtcctgagtcggctcattagatctctgctcctggcgtcccccgggcacctcgaggcccccggccggagcctctgctagtcaaatgccctcttccttcctgtgatgttctcactgcttccttagaaccgctgcagaagccaggcttgaccatctggaaccctgctggaaatcctagctctataatcatgtgattggcccagatcagtgttttctcacctcacttcccaccaagtgtctggagctgagtctgtgtatgctgtaatcacgaggtgtccacaggcgagatccccagtggttttgtttgcagaataaaaaggcctcagtgacccatgagaaaaaaaaaaaaaaaaaaa

SEQ ID NO: 10 - protein, 434 aa, Felis cattus
MSILKIHAREIFDSRGNPTVEVDLYTSKGLFRAAVPSGASTGIYEALELRDNDKTRYMGKGVSKAVEHINKTIAPALISKKLSVVEQEKIDKLMIEMDGTENKSKFGANAILGVSLAVCKAGAVEKGVPLYRHIADLAGNAEVILPVPAFNVINGGSHAGNKLAMQEFMILPVGAANFREAMRIGAEVYHNLKNVIKEKYGKDATNVGDEGGFAPNILENKEALELLKNAIGKAGYTDKVVIGMDVAASEFFRSGKYDLDFKSPDDPSRYITPDELANLYKSFIRDYPVVSIEDPFDQDDWEAWQKFTASAGIQVVGDDLTVTNPKRISKAVNERSCNCLLLKVNQIGSVTESLQACKLAQSNGWGVMVSHRSGETEDTFIADLVVGLCTGQIKTGAPCRSERLAKYNQILRIEEELGSKAKFAGRSFRNPLAK

SEQ ID NO: 11 - DNA, 24 bp, Artificial Sequence
  Primer
caccatgtctattctcaagatcca

SEQ ID NO: 12 - DNA, 24 bp, Artificial Sequence
  Primer
cttctttgttctccaggatgttag

SEQ ID NO: 13 - protein, 6 aa, Felis cattus
GVPLYR

SEQ ID NO: 14 - protein, 6 aa, Felis cattus
DNDKTR

SEQ ID NO: 15 - protein, 6 aa, Felis cattus
EIFDSR

SEQ ID NO: 16 - protein, 6 aa, Felis cattus
YDLDFK

SEQ ID NO: 17 - protein, 6 aa, Felis cattus
YNQILR

SEQ ID NO: 18 - protein, 8 aa, Felis cattus
IEEELGSK

SEQ ID NO: 19 - protein, 9 aa, Felis cattus
KVNVVEQEK

SEQ ID NO: 20 - protein, 9 aa, Felis cattus
SGKYDLDFK

SEQ ID NO: 21 - protein, 10 aa, Felis cattus
IGAEVYHNLK

SEQ ID NO: 22 - protein, 11 aa, Felis cattus
LMIEMDGTENK

SEQ ID NO: 23 - protein, 11 aa, Felis cattus
VNVVEQEKIDK

SEQ ID NO: 24 - protein, 12 aa, Felis cattus
YITPDQLADLYK

SEQ ID NO: 25 - protein, 13 aa, Felis cattus
YDLDFKSPDDPSR

SEQ ID NO: 26 - protein, 18 aa, Felis cattus
AAVPSGASTGIYEALELR

SEQ ID NO: 27 - protein, 19 aa, Felis cattus
DATNVGDEGGFAPNILENK

(19) United States
(12) Patent Application Publication
(10) Pub. No.: US 2006/0051744 A1
(43) Pub. Date: Mar. 9, 2006

(54) Feline infectious peritonitis (FIP) and systemic multi-organ coronavirus biomarkers and screening methods

(76) Inventors: Kimberly M. Austin, Manhattan, KS (US); Sanjay Kapil, Manhattan, KS (US); Jeong-Ki Kim, Manhattan, KS (US)

Correspondence Address: FISH & RICHARDSON P.C., PO BOX 1022, MINNEAPOLIS, MN, 55440-1022, US

(21) Appl. No.: 11/168,637

(22) Filed: Jun. 28, 2005

Related U.S. Application Data
(60) Provisional application No. 60/584,439, filed on Jun. 30, 2004.
(60) Provisional application No. 60/656,027, filed on Feb. 24, 2005.

(51) Int. Cl. C12Q 1/70; C12Q 1/68

(52) U.S. Cl. 435/5; 435/6

(57) ABSTRACT

Methods for screening for FIP infection or other multi-organ coronaviruses are disclosed, as well as isolated antibodies and kits useful for performing such methods. Biomarkers for multi-organ coronavirus infections include soluble enolase; antibodies to enolase; and circulating immune complexes that contain enolase. The methods find application in diagnosis, treatment, vaccine-development, and selection or breeding for disease-resistance.

### CROSS REFERENCE TO RELATED APPLICATION

This application claims priority to U.S. Provisional Application No. 60/584,439, filed Jun. 30, 2004 and U.S. Provisional Application No. 60/656,027, filed Feb. 24, 2005, both of which are incorporated by reference in their entirety herein.

### TECHNICAL FIELD

This invention relates to methods for screening for FIP infection, and more particularly to methods for screening for FIP that include detecting biological markers (biomarkers) associated with FIP infection. Such biomarkers include soluble enolase, antibodies to enolase, and circulating immune complexes (CICs) that include enolase as a component, and can be referred to as “Averill markers” or “Averill biomarkers.” Methods for screening for multi-organ coronaviruses, including SARS coronavirus, are also disclosed, as well as articles of manufacture (e.g., kits) and compositions useful for performing the methods.

### BACKGROUND

FIP is a fatal viral disease of wild and domestic cats caused by infection with a feline coronavirus (FCoV). Individuals of the Viverridae family (e.g., civet cat) and Felidae family, including those in the Felis, Panthera, Acinonyx, and Neofelis genera, can be infected with FCoV and exhibit a wide range of disease symptoms from fatal disease to simple seroconversion with no disease complications. It is widely believed, therefore, that there are at least two biotypes or strains of FcoV; both biotypes are considered to be antigenic group I type coronaviruses. The common, or enteric form, is known as Feline Enteric Coronavirus (FECV) and is nonpathogenic, causing mild enteritis, low-grade fever, anorexia, lethargy, and diarrhea. The pathogenic form is known as FIPV and demonstrates multi-organ pathology (e.g., commonly affected organs include the liver, lungs, brain, and eye). FIPV infection can manifest itself in what is termed a “dry” or “non-effusive” form or a “wet” or “effusive” form. The dry form can result in granulomas in affected organs such as the liver, kidneys, intestines, lymph nodes, eyes, and CNS, and can lead to jaundice (e.g., if the liver is involved); uveitis or retinitis; and nervous symptoms (e.g., wobbly gait). The wet form can lead to accumulation of fluid (e.g., ascites fluid) in the abdomen and/or chest (e.g., pleural, peritoneal, pericardial and/or renal subcapsular spaces). Fevers, weight loss, anorexia, and other non-specific symptoms can also be associated with both forms of the disease. Symptomatically, FIP can be confused with other multi-organ disorders or systemic disorders such as cardiac disease resulting in pleural effusion; lymphoma (e.g., in the kidneys); CNS tumors; or other respiratory or enteric diseases.

Because of the non-specific nature of many of the symptoms, accurate diagnosis of FIPV infection is difficult. Postmortem histopathological detection of granulomatous lesions is a definitive method, but provides no opportunity for treatment of the infected cat or early containment of an infectious outbreak (e.g., in a cattery or a shelter). For antemortem diagnosis, a variety of factors are typically taken together to support a diagnosis of FIP, including history of the cat; clinical signs; serology; clinical pathology; albumin and globulin levels and relative ratios of the two (e.g., hyperglobulinaemia); elevated serum liver enzyme and bilirubin levels; elevated fibrinogen levels; neutrophilia; lymphopenia; and proteinuria. Due to the non-specific nature of many of the factors, however, when a practitioner suspects FIPV infection, an accurate assessment of the stage and/or grade of the disease cannot be currently provided. It would be useful to have a more definitive ante-mortem method to diagnose FIP, as well as methods to provide an indication of the stage of the disease and probable disease course.

There have been some attempts to prepare vaccines against FIP CoV. For example, Primucell™ FIP vaccine, a commercially available vaccine, is a temperature-sensitive mutant of FIPV that replicates only in the upper respiratory tract of cats after vaccination. Because both FIP and SARS can demonstrate a more fulminant pathogenesis in seropositive animals that are subsequently exposed to the coronaviruses, however, the possibility of antibody-dependent enhancement (ADE) of FIPV or SARS CoV infection remains a concern in the preparation of coronavirus vaccines. (Martin Enserink, “One Year After Outbreak, SARS Virus Yields Some Secrets,” Science 304:1097 (2004).)

### SUMMARY

The disclosure is based on the discovery that FIPV infection can lead to an autoimmune pathology in infected individuals. While not being bound by theory, it is believed that interaction of the 3′UTR of FIPV viral RNA with one or more isoforms of the host cellular protein enolase leads to a conformational alteration of the host protein and the exposure of cryptic antigenic host domains, inducing an autoimmune response that includes anti-enolase antibody production, the accumulation of circulating immune complexes (CICs) in sera, bodily fluids, and organs such as the kidneys and lungs, and the release of free enolase into sera and bodily fluids due to lysis of target cells. The inventors have found that CICs of infected individuals can include enolase; antibodies, including antibodies specific for enolase; and viral FIPV RNA, including mRNAs or genomic RNA that include the 3′UTR of the viral RNAs.

The inventors have also found that sera or bodily fluids of infected individuals can also exhibit free (e.g., soluble and/or not associated with circulating immune complexes) enolase. Accordingly, methods for screening or diagnosing an individual suspected of having been exposed to or infected with FIPV are provided, which can include detecting one or more of the previously described biomarkers or components of CICs in a sample derived from the individual. The invention also provides isolated antibodies useful in the methods. Methods for screening an individual suspected of having a multi-organ coronavirus (e.g., SARS CoV), which may be a systemic multi-organ coronavirus, by detecting similar biomarkers are also disclosed, as well as articles of manufacture and kits useful for performing the described methods.

Accordingly, in one embodiment, a method for screening an individual of the Felidae family for FIP CoV exposure or infection includes determining whether or not a sample comprising circulating immune complexes from the individual includes enolase. The determining can include detection of the enolase, wherein the detection is indicative that the individual has been exposed to a virulent form of FIP CoV. The enolase can include the α-isoform and homo- or hetero-dimers of the α-isoform. The method can further include determining whether or not the sample comprises an antibody specific for enolase.

In certain cases, the method can include determining whether or not the sample comprises viral FIP RNA. The determining can include detecting the viral FIP RNA using a polynucleotide probe specific for the 3′UTR of the viral FIP RNA. The determining step can include detecting the enolase using a technique selected from the group consisting of: a western blot, a northwestern blot, an ELISA, a lateral flow immunoassay, an immunohistochemistry technique, and a protein sequencing method.

In some cases, a sample can include an antibody specific for enolase. In some cases, the sample comprises viral FIP RNA. A sample can be selected from the group consisting of serum, peritoneal fluid, thoracic fluid, cerebrospinal fluid, lymph, saliva, lachrymal fluid, aqueous or vitreous humor, ascites fluid, plasma, whole blood, a fresh biopsy sample, a fixed tissue sample, lavages, tracheal washings, and effusions of the individual.

In certain embodiments, a method for screening an individual of the Felidae family for FIP CoV exposure or infection can include determining whether or not a sample from the individual comprises an antibody specific for enolase. The sample can further include circulating immune complexes.

In some embodiments, a method for screening an individual of the Felidae family for FIP CoV exposure or infection is provided, which includes determining whether or not a sample from the individual comprises enolase, where the enolase is soluble enolase. For example, in some cases, soluble enolase is not associated with circulating immune complexes.

In another aspect, a method for screening an individual of the Felidae family for FIP CoV exposure or infection is provided, which includes determining whether or not a sample from the individual comprises circulating immune complexes, where the circulating immune complexes comprise enolase.

In yet another aspect, a method for determining whether or not a test vaccine for a multi-organ CoV is safe for administration is provided, which includes
(a) administering the test vaccine to an individual;
(b) determining whether or not an elevated level of antibodies specific for enolase is produced in the individual relative to a control individual not administered the test vaccine. An elevated level of antibodies specific for enolase can be indicative that the test vaccine is not safe for administration. The multi-organ CoV can be, for example, FIP, SARS, or a SARS-like virus.

In another embodiment, a method for determining whether or not a test vaccine for a multi-organ CoV is safe for administration includes:
(a) administering the test vaccine to an individual;
(b) determining whether or not an elevated level of free enolase in serum or bodily fluids of the individual is produced relative to a control individual not administered the test vaccine, where an elevated level of free enolase can be indicative that the test vaccine is not safe for administration.

In yet another embodiment, a method for determining whether or not a test vaccine for a multi-organ CoV is safe for administration includes:
(a) administering the test vaccine to an individual;
(b) determining whether or not an elevated level of CICs comprising enolase is produced in the individual relative to a control individual not administered the test vaccine, where an elevated level of CICs comprising enolase can be indicative that the test vaccine is not safe for administration.

Also provided is an isolated antibody specific for Felidae enolase. In some cases, the isolated antibody is not specific for human enolase. The antibody can be derived from an individual of the Felidae family. The antibody can be a component of a circulating immune complex. The Felidae enolase can be selected from the group consisting of the alpha-enolase isoform, the gamma-enolase isoform, alpha-alpha enolase, gamma-gamma enolase, alpha-gamma enolase, and mixtures thereof.

In another aspect, a method for isolating a circulating immune complex comprising enolase from an individual of the Felidae family includes contacting a biological fluid derived from the individual with polyethylene glycol in order to precipitate the circulating immune complex. The circulating immune complex can further comprises antibodies to enolase. The circulating immune complex can further comprise viral FIP RNA.

In yet another aspect, a method for screening an individual for multi-organ coronavirus exposure or infection is provided, which can include determining whether or not a sample comprising circulating immune complexes derived from the individual includes enolase. The multi-organ coronovirus can be selected from the group consisting of FIP, SARS, and a SARS-like virus.

In another embodiment, a method to determine if an individual that has been exposed to a multi-organ coronavirus is likely to develop a multi-organ pathology as a result of the exposure includes determining whether or not a sample comprising circulating immune complexes from the individual includes enolase.

Also provided is a method for screening a test agent to determine if it is useful for preventing or treating a multi-organ CoV infection. The method can include contacting a complex of enolase and multi-organ CoV RNA with the test agent; and determining if the test agent disrupts the complex. The multi-organ CoV RNA can include a 3′UTR of the multi-organ CoV RNA.

Also provided is an article of manufacture comprising an isolated antibody specific for Felidae enolase, where the isolated antibody is not specific for human enolase.

A method for evaluating if a test FIP vaccine has an increased tendency to induce an ADE response in a individual of the Felidae family is also provided, which can include:
(a) administering the test FIP vaccine to an individual of the Felidae family;
(b) determining whether or not, after the test FIP vaccine administration, the individual exhibits CICs comprising enolase in an elevated amount relative to a control individual not administered the test vaccine, where the elevated production of CICs can be indicative that the test FIP vaccine has an increased tendency to induce an ADE response.

A method for selecting an individual of the Felidae family for breeding is provided, which includes determining one or more of the following:
(a) whether or not free enolase is present in the individual's serum or bodily fluids;
(b) whether or not antibodies to enolase are present in the individual's serum, bodily fluids, or in CICs; and/or
(c) whether or not CICs comprising enolase are present in the individual, where a positive finding in (a), (b), or (c) is indicative that the individual is unsuitable for breeding.

In another aspect, a method for determining whether or not a test vaccine for a multi-organ CoV is safe for administration is provided, which can include administering the test vaccine to an individual; and determining whether or not an elevated level of antibodies capable of recognizing an N-terminal domain of enolase is produced in the individual relative to a level of the antibodies in a control individual not administered the test vaccine, where an elevated level of antibodies capable of recognizing the N-terminal domain of enolase is indicative that the test vaccine is not safe for administration, and where a non-elevated level is indicative that the test vaccine is safe for administration.

Also provided is a method of distinguishing a protective immunogenic FIP viral polypeptide from an FIP viral polypeptide that induces auto-antibodies to an auto-polypeptide in a mammal. The method can include contacting the FIP viral polypeptide with one or more antibodies capable of recognizing, independently, one or more candidate auto-polypeptides, and determining whether or not one or more of the antibodies recognizes the FIP viral polypeptide, where the recognition is indicative that the FIP viral polypeptide can induce the production of auto-antibodies to the auto-polypeptide in the mammal. In certain cases, the auto-antibodies are correlated with an increased tendency to induce an ADE response.

In another embodiment, a method of distinguishing a protective immunogenic domain of an FIP viral polypeptide from a domain of the FIP viral polypeptide that induces auto-antibodies to an auto-polypeptide in a mammal includes:
(a) contacting one or more candidate domains of the FIP viral polypeptide with one or more antibodies capable of recognizing, independently, one or more candidate auto-polypeptides, and
(b) determining whether or not one or more of the antibodies recognizes one or more of the candidate domains of the FIP viral polypeptide, where recognition of a candidate domain can be indicative that the candidate domain induces the production of auto-antibodies to the auto-polypeptide in the mammal.

Also provided is an isolated polynucleotide comprising a nucleic acid having 91% or higher sequence identity to SEQ ID NO:9. For example, an isolated polynucleotide can include a nucleic acid having 92%, 93%, 94%, 95%, 96%, 97%, 98%, 99%, or 100% sequence identity to SEQ ID NO:9. In some cases, the isolated polynucleotide is SEQ ID NO:9. Isolated polypeptides are also provided, which can include an amino acid sequence having 97% or higher sequence identity (e.g., 98%, 99%, or 100% sequence identity) to SEQ ID NO:10. In some cases, the isolated polypeptide is SEQ ID NO:10.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the sequence of the 3′untranslated region (UTR) riboprobe (below; SEQ ID NO:3; see also Example 1) aligned with feline coronavirus mRNA for 7a and 7b protein (3′untranslated region) strain FIPV UCD2 (above; SEQ ID NO:4).

FIG. 2 demonstrates one dimensional polyacrylamide gel electrophoresis of feline tissue proteins followed by northwestern analysis with a 3′UTR FIPV riboprobe. Lane 1, CrFK cell lysate; Lane 2, Brain cat1; Lane 3, Brain cat2; Lane 4, Spleen cat1; Lane 5, Spleen cat2; Lane 6, Spleen cat3; Lane 7, Spleen cat4; Lane 8, Pancreas cat1; Lane 9, Pancreas cat2; Lane 10, Pancreas cat3.

FIG. 3 demonstrates the effect of ions on FIPV RNA-binding complex formation with enolase in susceptible and nonsusceptible cell lines. Increasing amounts of NaCl were added to the hybridization buffer. Total cell protein lysates were incubated with 3′UTR FIPV (−)strand RNA. The complex was resolved on 12% SDS-PAGE. Lane 1, Meat Animal Research Center (MARC) cell lysate with 50 mM NaCl in SBB buffer; Lane 2, MARC with 100 mM NaCl in SBB buffer; Lane 3, MARC with 200 mM NaCl in SBB buffer; Lane 4, MARC with 50 mM KCl and 50 mM NaCl in SBB buffer; Lane 5, Madin-Darby Bovine Kidney (MDBK) cell lysate with 50 mM NaCl in SBB buffer; Lane 6, MDBK with 100 mM NaCl in SBB buffer; Lane 7, MDBK with 200 mM NaCl in SBB buffer; Lane 8, MDBK with 50 mM KCl and 50 mM NaCl in SBB buffer; Lanes 9-12 Crandel Feline Kidney (CrFK) cell lysate with buffers of same composition as Lanes 1-4 separately; Lanes 13-15, Swine Testicle (ST) cell lysate with composition of Lanes 1-4 for 13-15 respectively.

FIG. 4 demonstrates the results from two-dimensional northwestern blot analysis of the interaction between the 3′-untranslated region FIPV RNA with Crandell Feline Kidney (CRFK) total cell lysates. The high affinity binding between isoforms of enolase and FIPV RNA is evident.

FIG. 5a demonstrates a 3′UTR FIPV-probed Northwestern blot of pathologically-confirmed cases of wet and dry FIP infection in various tissues. Differential binding can be seen between the dry and wet forms. Lane 1, Meat Animal Research Center total cell lysate; Lane 2, Crandell Feline Kidney total cell lysate; Lane 3, Spleen; Lane 4, Liver Dry FIP; Lane 5, Liver Wet FIP; Lane 6, Lung Dry FIP; Lane 7, Lung Wet FIP; Lane 8, Lymph Node Dry FIP; Lane 9, Lymph Node Wet FIP; Lane 10, Spleen Dry FIP; Lane 11, Spleen Wet FIP; Lane 12, Heart Dry FIP; Lane 13, Heart Wet FIP; Lane 14, Brain Dry FIP; Lane 15, Brain Wet FIP; Lane 16, Small Intestine Dry FIP; Lane 17, Small Intestine Wet FIP; Lane 18, Spleen Dry FIP; Lane 19, Spleen Wet FIP; Lane 20, Kaleidoscope Protein Marker (BioRad, Hercules, Calif.).

FIG. 5b demonstrates a feline tissue western blot performed on the same northwestern nitrocellulose blot as shown in FIG. 5a. Feline tissues of both pathologically confirmed cases of wet and dry feline infectious peritonitis were run on a 10% SDS PAGE gel and a western blot was performed with α-enolase antibody (Santa Cruz Biotechnology, Santa Cruz, Calif.). Two bands are present. Lane 1, Meat Animal Research Center total cell lysate; Lane 2, Crandell Feline Kidney total cell lysate; Lane 3, Spleen; Lane 4, Liver Dry FIP; Lane 5, Liver Wet FIP; Lane 6, Lung Dry FIP; Lane 7, Lung Wet FIP; Lane 8, Lymph Node Dry FIP; Lane 9, Lymph Node Wet FIP; Lane 10, Spleen Dry FIP; Lane 11, Spleen Wet FIP; Lane 12, Heart Dry FIP; Lane 13, Heart Wet FIP; Lane 14, Brain Dry FIP; Lane 15, Brain Wet FIP; Lane 16, Small Intestine Dry FIP; Lane 17, Small Intestine Wet FIP; Lane 18, Spleen Dry FIP; Lane 19, Spleen Wet FIP; Lane 20, Kaleidoscope Protein Marker (BioRad, Hercules, Calif.).

FIG. 6a demonstrates the MALDI-TOF mass spectra obtained from a Micromass TofSpec SE instrument following tryptic-digestion of excised spots from two-dimensional SDS-PAGE gels. To attain a high level of accuracy for peptide mass searching, internal calibrants of 50 fmol bradykinin, which has a protonated, monoisotopic mass of 1060.57, and 150 fmol ACTH clip, which has a protonated, monoisotopic mass of 2465.2, were added to the sample.

FIG. 6b demonstrates ProFound database results; ProFound relies on the NCBI non redundant database for spectra analysis. Searching is carried out with a mass range that extends from 50% to 150% of the molecular weight (MW) estimated from SDS PAGE. The top score on the ProFound search was 1.0e+00 to alpha-enolase. A second ProFound search was performed after deleting masses which matched, with no additional proteins being identified.

FIG. 7 is the cDNA sequence of feline α-enolase (SEQ ID NO: 9). Start codon (ATG) and stop codon (TAA) are underlined. A poly(A) sequence (19 Adenines) was found at the end of sequence. Between TAA and poly(A) is the 3′ Untranslated Region (3′UTR). The 7-nucleotide sequence before start codon is part of the 5′ Untranslated Region (5′UTR). There are 1,305 nucleotides from the start codon to the stop codon, inclusive.

FIG. 8 is the amino acid sequence of feline α-enolase (SEQ ID NO: 10). It contains 434 amino acids encoded by the cDNA sequence shown in FIG. 7.

FIG. 9 shows the effect of pH 2.0 on α-enolase antibody titers. After treatment with pH 2.0, the OD reading of specific pathogen-free negative control, was unchanged. However, the positive samples showed higher OD reading. Serum for sample 1 and 2 was from an unknown clinical case that was tested positive only after acidification. Serum for sample 3 and 4 was from a clinically normal, healthy cat that served as a negative control. Serum for sample 5 and 6 was from an FIPV-infected cat. Samples 1, 3 and 5 were treated with acid. Samples 2, 4 and 6 were untreated.

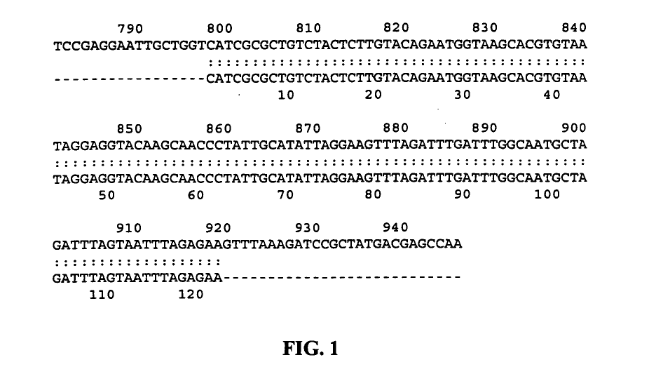

### DETAILED DESCRIPTION

In general, the invention provides methods and materials related to screening for multi-organ coronavirus (CoV) infections, including, without limitation, FIP CoV and SARS CoV, and multi-organ autoimmune diseases associated with such CoVs. As certain coronaviruses, including SARS and SARS-like coronaviruses, are believed to have jumped from animals, such as palm civets, ferrets, and birds, to humans, the invention thus provides, among other things, a useful analytical tool in tracking such multi-organ CoV infections. Methods for evaluating vaccines to treat or to prevent multi-organ CoVs are also disclosed, as well as methods for preventing or treating multi-organ CoV infections and CoV-associated autoimmune conditions.

Specifically, the invention provides methods and materials related to screening an animal, such as a mammal or a bird for exposure to or infection by a multi-organ coronavirus. A mammal for screening can be, without limitation, a human, civet cat, palm civet, dog, cat (e.g., domestic, wild, and large cats), raccoon, ferret, skunk, mink, weasel, ermine, polecat, marten, badger, otter, river otter, horse, cow, goat, sheep, pig, and rodent (e.g., mouse, rat). A mammal can be an individual of the Felidae, Viverridae, or Mustilidae families. A bird can be any bird, including without limitation, a chicken, duck, goose, pigeon, turkey, pheasant, grouse, sparrow, starling, or jay. A multi-organ coronavirus can affect more than one organ (e.g., two or more of the eyes, brain, intestines, kidney, liver, lungs, and macrophages) and may be systemic. For example, the invention provides methods and materials for screening an individual of the Felidae family suspected of having been exposed to FIP CoV for FIP CoV infection. Members of the Felidae family include, without limitation, wild and domestic cats, pallas cats, bobcats, lynx, mountain lions, cougars, pumas, lions, leopards, tigers, white tigers, leopards, and snow leopards. A method can include, determining, among other things, whether or not a sample from that individual that includes circulating immune complexes contains enolase. Articles of manufacture for use in the methods, including multiplex and/or panel kits and assays, are also described.

For the purpose of this invention, the term “multi-organ coronavirus” refers to a coronavirus that exhibits a multi-organ pathology. Organs affected can include, without limitation, the lung, intestine, brain, kidney, liver, eye (e.g., retina), and macrophages. In certain cases, a systemic pathology can result. Examples of multi-organ coronaviruses include, without limitation, FIP CoV, SARS CoV, and SARS-like coronaviruses (e.g., Pearson, Helen “SARS may not be alone: antibodies to a SARS-like virus hint at repeated infections,” Nature Science Update (Jan. 15, 2004), available at www.nature.com). Certain multi-organ coronaviruses are referred to as antigenic group I coronaviruses. Certain multi-organ coronaviruses, such as SARS CoV and FIP CoV, can exhibit amino acid similarity in regions of their spike protein amino acid sequences (e.g., about 20 to about 50% amino acid identity).

The term “autoimmune condition associated with a coronavirus” refers to any condition resulting from a mammal's body tissue being attacked by that mammal's own immune system after exposure to or infection by the coronavirus. For example, a patient with an autoimmune condition can have antibodies (e.g., anti-enolase antibodies) in their blood that target their own body tissues.

As used herein, “enolase” can refer to one or more of the monomeric isoforms of enolase, including the alpha and gamma monomeric isoforms, as well as homo-dimers (e.g., alpha-alpha, gamma-gamma) or hetero-dimers (e.g., alpha-gamma). In certain instances, a specific reference to a particular monomeric isoform (e.g., alpha enolase, gamma enolase) or a particular dimeric form (e.g, alpha-gamma, alpha-alpha, gamma-gamma) may be used. Enolase 1 is a cytoplasmic, alpha-alpha homodimeric protein that is found in most tissues. Enolase 2 or neuronal enolase is a cytoplasmic gamma-gamma homodimer found in mature neurons and cells of neuronal origin. Neuron Specific Enolase (NSE) refers to a mixture of gamma-gamma homodimers and alpha-gamma heterodimers.

The term “specific for enolase” with respect to an antibody refers to the ability of an antibody to bind to and recognize at least one isoform of enolase. For example, an antibody can be specific for alpha-enolase, e.g., can bind to and recognize an epitope on alpha-enolase. Alternatively, an antibody can be specific for gamma-enolase, e.g., can bind to and recognize an epitope on gamma-enolase. In certain cases, an antibody specific for alpha enolase can be used to detect alpha-alpha enolase homodimers and/or alpha-beta and alpha-gamma heterodimers. In other cases, an antibody specific for gamma enolase can be used to detect gamma-gamma homodimers and/or alpha-gamma and beta-gamma heterodimers. In certain instances, an antibody can be “specific for” more than one isoform of enolase, e.g., it can bind to and recognize both the alpha and gamma isoforms. In such cases, the antibody can bind to and recognize one isoform to a similar or a different degree relative to another isoform. In other instances, an antibody can be “specific for” only one isoform of enolase, with minimal or no recognition of the other isoforms.

As used herein “soluble enolase” and “free enolase” are used interchangeably and refer to enolase that is not contained within a cellular membrane (e.g., not cytoplasmic) and/or is not associated with circulating immune complexes. Thus, soluble enolase can be detected in centrifuged, cell-free preparations of, for example, biological fluids.

Typically, samples to be evaluated for the presence of free enolase do not include cellular components or CICs or will have had cellular and CIC components largely removed. For example, biological fluid samples such as serum, peritoneal fluid, thoracic fluid, cerebrospinal fluid, lymph, saliva, lachrymal fluid, aqueous or vitreous humor, ascites fluid, plasma, lavages, tracheal washings, and effusions can be treated to remove cellular content and examined for the presence and amount of free enolase, such as by centrifugation at about 1000 g or more or through filtration (e.g., through a 0.1 micron filter).

Isolated α-Enolase Polynucleotides and Polypeptides

Disclosed herein are isolated polynucleotides and polypeptides, which can be useful for various applications. For example, isolated polynucleotides and polypeptides can be used in methods of screening for FIP infection; in methods for determining whether or not a test vaccine for a multi-organ coronavirus is safe; for producing an antibody specific for Feline α-enolase; and for selection or breeding for disease-resistance.

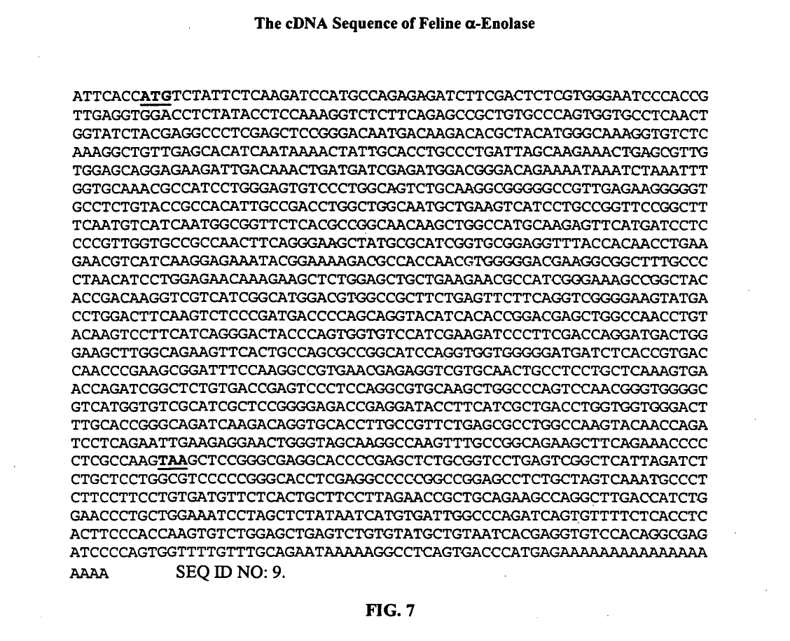

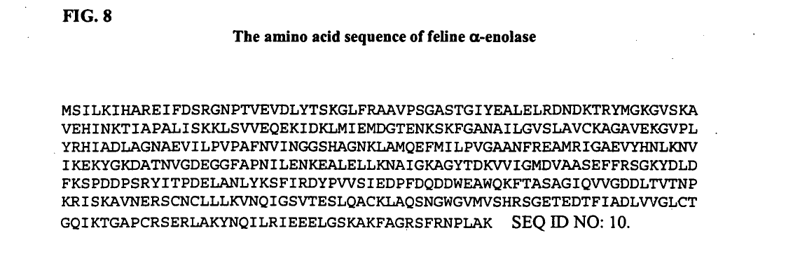

An isolated polynucleotide disclosed herein can include a nucleic acid having 91% or higher sequence identity to SEQ ID NO:9. SEQ ID NO:9 is the cDNA sequence for Feline α-enolase, which was determined as shown in Example 8 and is set forth FIG. 7. In certain cases, an isolated polynucleotide can include a nucleic acid that is 92%, 93%, 94%, 95%, 96%, 97%, 98%, 99% or 100% identical to SEQ ID NO:9. In other cases, an isolated polynucleotide has the sequence of SEQ ID NO:9.

An isolated polynucleotide can include a nucleic acid encoding a polypeptide having 97% or higher sequence identity to SEQ ID NO:10. For example, a nucleic acid can encode a polypeptide that is 98%, 99% or 100% identical to SEQ ID NO:10.

An isolated polypeptide can include an amino acid sequence having 97% or higher sequence identity to SEQ ID NO:10. For example, the amino acid sequence can be 98%, 99% or 100% identical to SEQ ID NO:10.

As used herein, the terms “nucleic acid” or “polynucleotide” are used interchangeably and refer to both RNA and DNA, including cDNA, genomic DNA, synthetic (e.g., chemically synthesized) DNA, and DNA (or RNA) containing nucleic acid analogs. Polynucleotides can have any three-dimensional structure, and can be in the sense or antisense orientation. Nonlimiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

Isolated nucleic acid molecules can be produced by standard techniques. For example, polymerase chain reaction (PCR) techniques can be used to obtain an isolated nucleic acid containing a nucleotide sequence described herein. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length (e.g., 10, 15, 20, 25, 27, 34, 40, 45, 50, 52, 60, 65, 70, 75, 82, 90, 102, 150, 200, 250 nucleotides in length). General PCR techniques are described, for example in PCR Primer: A Laboratory Manual, Ed. by Dieffenbach, C. and Dveksler, G, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis, 1992, Genetic Engineering News, 12: 1; Guatelli et al., 1990, Proc. Natl. Acad. Sci. USA, 87: 1874-1878; and Weiss, 1991, Science, 254: 1292.

Isolated nucleic acids of the invention also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids of the invention also can be obtained by mutagenesis. For example, a reference nucleic acid sequence be mutated using standard techniques including oligonucleotide-directed mutagenesis and site-directed mutagenesis through PCR. Short Protocols in Molecular Biology, Chapter 8, Green Publishing Associates and John Wiley & Sons, Edited by Ausubel, F. M et al., 1992.

Nucleic acid analogs can be modified at the base moiety, sugar moiety, or phosphate backbone to improve, for example, stability, hybridization, or solubility of the nucleic acid. Modifications to the backbone include the use of uncharged linkages (e.g., methyl phosphonates, phosphotriesters, phosphamidates, carbamates, etc.) and charged linkages (e.g., phosphorothioates, phosphorodithioates, etc.). Modifications to the backbone can also incorporate peptidic linkages, e.g., to result in a PNA-type linkage. Modifications at the base moiety include deoxyuridine for deoxythymidine, and 5-methyl-2′-deoxycytidine or 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. Summerton and Weller, Antisense Nucleic Acid Drug Dev. (1997) 7(3):187-195; and Hyrup et al. (1996) Bioorgan. Med. Chem. 4(1):5-23. In addition, the deoxyphosphate backbone can be replaced with, for example, a phosphorothioate or phosphorodithioate backbone, a phosphoroamidite, or an alkyl phosphotriester backbone. The nucleic acid can be double-stranded or single-stranded (i.e., a sense or an antisense single strand).

As used herein, “isolated,” when in reference to a nucleic acid or polynucleotide, refers to a nucleic acid or polynucleotide that is separated from other nucleic acid or polynucleotide molecules that are present in a genome, e.g., a cat genome, including nucleic acids or polynucleotides that normally flank one or both sides of the nucleic acid or polynucleotide in the genome. The term “isolated” as used herein with respect to nucleic acids or polynucleotides also includes any non-naturally-occurring sequence, since such non-naturally-occurring sequences are not found in nature and do not have immediately contiguous sequences in a naturally-occurring genome.

An isolated nucleic acid or polynucleotide can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

The term “polypeptide” is used in its broadest sense to refer to a compound of two or more subunit amino acids, amino acid analogs, or other peptidomimetics. The subunits may be linked by peptide bonds or other bonds, for example, ester, ether, etc. The term “amino acid” refers to either natural and/or unnatural or synthetic amino acids, including the D/L optical isomers. Full-length proteins, analogs, mutants, and fragments thereof are encompassed by this definition.

By “isolated,” with respect to a polypeptide, it is meant that the polypeptide is separated to some extent from the cellular components with which it is normally found in nature. An isolated polypeptide can yield a single major band on a non-reducing polyacrylamide gel. In certain cases, a polypeptide is “purified.” The term “purified” as used herein preferably means at least about 75% by weight or more (e.g., at least 80%, 85%, 90%, 95%, 97%, 98%, 99%, or 100%) of polypeptides of the same type are present relative to all polypeptides in, e.g., a mixture. Isolated polypeptides can be obtained, for example, by extraction from a natural source, by chemical synthesis, or by recombinant production in a host cell or transgenic plant.

To recombinantly produce polypeptides, a nucleic acid sequence containing a nucleotide sequence encoding the polypeptide of interest can be ligated into an expression vector and used to transform a bacterial, eukaryotic, or plant host cell (e.g., insect, yeast, mammalian, or plant cells). In bacterial systems, a strain of Escherichia coli such as BL-21 can be used. Suitable E. coli vectors include the pGEX series of vectors that produce fusion proteins with glutathione S-transferase (GST). Depending on the vector used, transformed E. coli are typically grown exponentially, then stimulated with isopropylthiogalactopyranoside (IPTG) prior to harvesting. In general, expressed fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety. Alternatively, 6× His-tags can be used to facilitate isolation.

In eukaryotic host cells, a number of viral-based expression systems can be utilized to express polypeptides. A nucleic acid encoding a polypeptide of the invention can be cloned into, for example, a baculoviral vector such as pBlueBac (Invitrogen, Carlsbad, Calif.) and then used to co-transfect insect cells such as Spodoptera frugiperda (Sf9) cells with wild type DNA from Autographa californica multiply enveloped nuclear polyhedrosis virus (AcMNPV). Recombinant viruses producing polypeptides of the invention can be identified by standard methodology.

Mammalian cell lines that stably express polypeptides can be produced by using expression vectors with the appropriate control elements and a selectable marker. For example, the pcDNA3 eukaryotic expression vector (Invitrogen, Carlsbad, Calif.) is suitable for expression of polypeptides in cell such as, Chinese hamster ovary (CHO) cells, COS-1 cells, human embryonic kidney 293 cells, NIH3T3 cells, BHK21 cells, MDCK cells, ST cells, PK15 cells, or human vascular endothelial cells (HUVEC). In some instances, the pcDNA3 vector can be used to express a polypeptide in BHK21 cells, where the vector includes a CMV promoter and a G418 antibiotic resistance gene. Following introduction of the expression vector, stable cell lines can be selected, e.g., by antibiotic resistance to G418, kanamycin, or hygromycin. Alternatively, amplified sequences can be ligated into a mammalian expression vector such as pcDNA3 (Invitrogen, San Diego, Calif.) and then transcribed and translated in vitro using wheat germ extract or rabbit reticulocyte lysate.

In yet other cases, plant cells can be transformed with a recombinant nucleic acid construct to express the polypeptide. The polypeptide can then be extracted and purified using techniques known to those having ordinary skill in the art.

Methods to Determine Percent Sequence Identity

Percent sequence identity is calculated by determining the number of matched positions in aligned nucleic acid or polypeptide sequences, dividing the number of matched positions by the total number of aligned nucleotides or amino acids, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (www.fr.com/blast) or the U.S. government's National Center for Biotechnology Information web site (www.ncbi.nlm.nih.gov). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. The following command will generate an output file containing a comparison between two sequences: C:\B12seq -i c:\seq1.txt -j c:\seq2.txt -p blastn -o c:\output.txt -q -1-r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100.

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Methods for Screening for FIP

As described herein, the invention is based on the finding that cats that have been exposed to FIP CoV produce circulating immune complexes that contain, among other things, enolase, viral FIP RNA, and antibodies specific for enolase. In certain circumstances, the enolase can include alpha and/or gamma isoforms of enolase (e.g., alpha monomer, gamma monomer, alpha-alpha homodimers, gamma-gamma homodimers, alpha-gamma heterodimers). In addition, the inventors have found that exposed cats also produce antibodies specific for enolase (e.g., antibodies specific for alpha enolase and/or antibodies specific for gamma enolase), and exhibit soluble isoforms of enolase (e.g., soluble alpha enolase, soluble gamma enolase, homo- or heterodimers of the same, and neuron specific enolase) in biological fluid samples, e.g., ascites fluid.

Accordingly, a method for screening an individual of the Felidae family for FIP CoV exposure or infection can include one or more of the following steps, either alone or in any combination or order:
(a) determining whether or not a sample that includes circulating immune complexes from the individual includes enolase;
(b) determining whether or not a sample (e.g., a biological fluid sample) from the individual includes soluble enolase;
(c) determining whether or not a sample from the individual, including a sample that contains CICs, includes antibodies specific for enolase; and/or
(d) determining whether or not a sample that includes circulating immune complexes from the individual includes FIP RNA. An FIP RNA can be genomic or mRNA, and can include the common 3′UTR of FIP RNAs.

A positive finding in one or more of the above steps is indicative that the individual has been exposed to FIP CoV, including a virulent form of FIP CoV. A positive finding can be indicative that the individual is likely to demonstrate a pathology associated with FIP, including a pathology associated with the wet or dry forms of FIP. A positive finding can also be indicative that the individual has a heightened risk of developing FIP in the future relative to a control individual (e.g., an individual who has not been exposed, who is uninfected, or who has been successfully vaccinated).

The level of enolase or antibody to enolase detected can be elevated relative to a corresponding reference or control level, e.g., a level from an uninfected or unexposed individual. For example, an elevated level of enolase or an enolase antibody can be 0.5, 1, 2, 3, 4, 5, 6, 7, 8, 9, 10, 15, 20, or more times greater than the reference level. In addition, a reference level can be any amount. For example, a reference level can be zero. In this case, any detected level of enolase or enolase antibodies greater than zero would be an elevated level.

Any method can be used to determine whether or not a sample, including a sample that includes CICs, includes enolase; free enolase; or antibodies to enolase. Typically, the enolase or the antibodies to enolase are detected by methods known to those having ordinary skill in the art. Methods of detection and/or quantification can be direct or competitive and steady-state or kinetic in nature. For example, methods for detection include, without limitation, immunohistochemistry methods, Western blots, Northwestern blots, ELISAs, protein sequencing methods, lateral flow immunoassay techniques (e.g., Al-Yousif et al., Clinical and Diagnostic Laboratory Immunology, May 2002, pages 723-724), agglutination tests (e.g., Al-Yousif et al., Clinical and Diagnostic Laboratory Immunology, May 2001, pages 496-498), radial immunodiffusion techniques, and immunofluorescence techniques, as described more fully below.

Specifically, enolase can be detected by detecting, without limitation, native, mutant, or truncated forms of the enolase protein. Antibodies recognizing enolase or specific for enolase can be detected by detecting any antibody that recognizes any epitope within enolase. Such antibodies can be polyclonal or monoclonal, and can be of any immunoglobulin class (e.g., IgA, IgD, IgE, IgG, or IgM) or subclass (e.g., IgG1, IgG2, IgG3, or IgG4).

In certain methods, enolase or antibodies recognizing enolase can be used to determine whether or not a sample contains antibodies recognizing enolase or enolase, respectively. For example, enolase can be used to determine whether or not a sample contains antibodies that recognize an epitope or combination of epitopes within enolase. Enolase is a highly conserved protein among both isoforms (e.g., alpha, beta, gamma) and species (e.g., human, yeast, mouse). For example, human alpha, beta, and gamma enolase demonstrate about 80% or higher amino acid sequence identity. In addition, antibodies specific for a particular species' enolase (e.g., antibodies to human alpha-enolase) can be cross-reactive with enolase from another species (e.g., Felidae alpha-enolase). Thus, in certain circumstances, a particular isoform of enolase from a particular species can be used to detect antibodies specific for an isoform of enolase from another species, and vice-versa.

An enolase isoform, for example, can be immobilized on a column matrix, and an antibody-containing fluid (e.g., serum) can be screened for the presence or absence of antibodies that have affinity for that isoform. As indicated previously, as enolase is highly conserved among species and as enolase antibodies can demonstrate cross-reactivity among species, the enolase isoform that is immobilized need not be from the same species as the antibodies to enolase to be detected, although in some cases it can be.

For example, human alpha-enolase or yeast alpha-enolase can be immobilized on a column matrix in order to screen for the presence or absence of Felidae antibodies that have affinity for alpha-enolase. Alternatively, wells on a microtiter plate can be coated, independently, with one or more enolase isoforms and/or dimeric forms, and an antibody-containing fluid (e.g., serum) can be screened by ELISA techniques for the presence or absence of antibodies that recognize a specific isoform or combination of isoforms. In addition, one or more isoforms of enolase can be used in a radioimmunoassay to determine whether or not a sample contains antibodies specific for the one or more isoforms.

Antibodies that recognize enolase can be used to determine whether or not a sample contains enolase, including soluble enolase. Anti-enolase antibodies can, for example, be used to detect enolase in a sample. For example, anti-alpha-enolase antibodies can be used to detect alpha-enolase in a sample. Similarly, anti-gamma enolase antibodies can be used to detect gamma-enolase or neuron specific enolase in a sample. As indicated previously, antibodies to enolase demonstrate cross-reactivity among species. Accordingly, antibodies employed to detect enolase need not necessarily have been raised against (or prepared against) enolase from the same species as to be detected, although in certain circumstances such antibodies may be used. In certain circumstances, anti-human alpha-enolase antibodies can be used to detect Felidae alpha-enolase. In other cases, an antibody that is specific for a particular species' enolase (or enolase isoform) but that is not specific for another species' enolase (or enolase isoform) may be used. For example, an antibody that is specific for Felidae alpha-enolase but not specific for human alpha-enolase may be used.

Any of the methods indicated previously can be used to detect enolase including, without limitation, western blot, ELISA, and immunohistochemistry techniques. For example, a sample (e.g., a biological fluid such as CSF, plasma, serum, lymph, vitreous humour, ascites fluid) from a mammal can be centrifuged to remove cell debris. The sample can be procured, transported, and/or stored in a manner that does not result in hemolysis, particularly in screens to determine the presence or absence of soluble enolase. In healthy cells, enolase is cytoplasmic and thus should typically only be found in extracellular fluids (e.g., serum, plasma) in conditions of abnormal pathology. The polypeptides in the resulting supernatant can be electrophoretically separated in a gel under non-denaturing or denaturing conditions. Once separated, the polypeptides can be electrophoretically transferred to a suitable substrate (e.g., a nitrocellulose membrane). The presence or absence of enolase in the sample can be determined by processing the polypeptide-containing substrate with primary antibodies that are known to recognize one or more isoforms of enolase using standard western blotting techniques known in the art. Alternatively, wells on a microtiter plate can be coated, independently, with one or more antibodies, that recognize enolase or particular enolase isoforms, and a protein containing fluid (e.g., serum or ascites fluid) can be screened by ELISA techniques for the presence or absence of a particular isoform or combination of isoforms.

Preferred methods of detecting or measuring enolase or an antibody to enolase in biological fluid samples employ antibodies (e.g., polyclonal antibodies or monoclonal antibodies (mAbs)) that bind specifically to enolase. In such methods, the antibody itself or a secondary antibody that binds to it can be detectably labeled. Alternatively, the antibody can be conjugated with biotin, and detectably labeled avidin (a protein that binds to biotin) can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. In such assays, the presence of enolase in atypical locations can be assessed.

Methods of detection basically involve contacting a sample of interest with an antibody that binds to enolase, and testing for binding of the antibody to a component of the sample. In such assays, the antibody need not be detectably labeled and can be used without a second antibody that binds to enolase. For example, by exploiting the phenomenon of surface plasmon resonance, an antibody specific for enolase bound to an appropriate solid substrate is exposed to the sample. Binding of enolase to the antibody on the solid substrate results in a change in the intensity of surface plasmon resonance that can be detected qualitatively or quantitatively by an appropriate instrument, e.g., a Biacore apparatus (Biacore International AB, Rapsgatan, Sweden). In other cases, the enzymatic activity of enolase can be detected using functional activity assays known to those having ordinary skill in the art.

Moreover, assays for detection of enolase or an enolase antibody in a sample (e.g., a biological fluid sample) can involve the use, for example, of: (a) a single enolase-specific antibody that is detectably labeled; (b) an unlabeled enolase-specific antibody and a detectably labeled secondary antibody; or (c) a biotinylated enolase-specific antibody and detectably labeled avidin. In addition, combinations of these approaches (including “multi-layer” assays) familiar to those in the art can be used to enhance the sensitivity of assays. In these assays, the sample or an (aliquot of the sample) suspected of containing enolase can be immobilized on a solid substrate such as a nylon or nitrocellulose membrane by, for example, “spotting” an aliquot of the liquid sample or by blotting of an electrophoretic gel on which the sample or an aliquot of the sample has been subjected to electrophoretic separation. The presence or amount of enolase on the solid substrate is then assayed using any of the above-described forms of the enolase-specific antibody and, where required, appropriate detectably labeled secondary antibodies or avidin.

Methods for detecting enolase or enolase antibodies can include “sandwich” assays. In these sandwich assays, instead of immobilizing samples on solid substrates by the methods described above, any enolase that may be present in a sample can be immobilized on the solid substrate by, prior to exposing the solid substrate to the sample, conjugating a second (“capture”) enolase-specific antibody (polyclonal or mAb) to the solid substrate by any of a variety of methods known in the art. In exposing the sample to the solid substrate with the second enolase-specific antibody bound to it, enolase in the sample (or sample aliquot) will bind to the second enolase-specific antibody on the solid substrate. The presence or amount of enolase bound to the conjugated second enolase-specific antibody is then assayed using a “detection” enolase-specific antibody by methods essentially the same as those described above using a single enolase-specific antibody. It is understood that in these sandwich assays, the capture antibody should not bind to the same epitope (or range of epitopes in the case of a polyclonal antibody) as the detection antibody. Thus, if a mAb is used as a capture antibody, the detection antibody can be either: (a) another mAb that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope that is either completely physically separated from or partially overlaps with any of the epitopes to which the capture polyclonal antibody binds; or (b) a polyclonal antibody that binds to epitopes other than or in addition to that to which the capture polyclonal antibody binds. Assays which involve the used of a capture and detection antibody include sandwich ELISA assays, sandwich Western blotting assays, and sandwich immunomagnetic detection assays.

Suitable solid substrates to which the capture antibody can be bound include, without limitation, the plastic bottoms and sides of wells of microtiter plates, membranes such as nylon or nitrocellulose membranes, and polymeric (e.g., without limitation, agarose, cellulose, or polyacrylamide) beads or particles. It is noted that enolase-specific antibodies bound to beads or particles can also be used for immunoaffinity purification of enolase.

Methods of detecting or for quantifying a detectable label depend on the nature of the label and are known in the art. Appropriate labels include, without limitation, radionuclides (e.g., ¹²⁵I, ¹³¹I, ³⁵S, ³H, ³²P, ³³P, or ¹⁴C), fluorescent moieties (e.g., fluorescein, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). The products of reactions catalyzed by appropriate enzymes can be, without limitation, fluorescent, luminescent, or radioactive or they may absorb visible or ultraviolet light. Examples of detectors include, without limitation, x-ray film, radioactivity counters, scintillation counters, spectrophotometers, colorimeters, fluorometers, luminometers, and densitometers.

A sample can be ante- or post-mortem and can include tissues, fluids, or both. A fluid sample (e.g., a biological fluid sample) can include serum, peritoneal fluid, thoracic fluid, ascites fluid, cerebrospinal fluid, lymph, saliva, lachrymal fluid, aqueous or vitreous humor, plasma, whole blood, effusions, lavages, or tracheal washings. In certain cases, tissue samples can be used, such as biopsy samples (e.g., renal or hepatic biopsies) or fixed tissue samples (e.g., formalin fixed samples). Samples can be collected and stored using techniques known to those having ordinary skill in the art. Once obtained, a sample can be manipulated. For example, serum can be separated from the other blood components in a peripheral blood sample by centrifugation. For assays designed to screen for the presence of free enolase, cellular content and/or CICs can be removed from the sample, and the sample can be collected in a manner that does not result in hemolysis by methods known to those having ordinary skill in the art.

In certain cases, a sample can include circulating immune complexes. Enolase and/or antibodies to enolase can be associated with CICs, e.g., CICs precipitated as described below. CICs can include, among other things, antibody-antigen complexes (e.g. anti-enolase antibody-enolase complexes), complement, and/or viral RNA.

Circulating immune complexes can be isolated from a biological fluid sample (e.g., sera, ascites fluid, peritoneal fluid) from an individual. CICs can be isolated from a biological fluid by precipating CICs, for example, using cryoprecipitation, ultracentrifugation, sucrose gradient density centrifugation, gel filtration, ultrafiltration, electrophoresis, electrofocusing, and sedimentation. For example, CICs can be precipitated by contacting a sample with polyethylene glycol (PEG), such as with PEG 6000 or PEG 8000, followed by centrifugation. In certain cases, CICs can be precipitated and/or denatured with boric acid, Tris-HCl, Glycine HCl, or Guanidine HCl. A precipitant such as PEG 6000 or 8000 can be used at a percentage by weight of the solution of from about 2% to about 8% (e.g., about 3, 3.5, 4, 4.5, 5, 6, 7, 7.5% by weight). A fluid sample can be incubated with a precipitant, such as a PEG solution, at a temperature from about 2° C. to about 10° C., and for a time period from about 10 h. to about 30 h. (e.g., about 10, 15, 18, 20, 25, or 28 h.). Centrifugation can be at about 1500-2500×g (e.g., about 1700, 1800, 1900, 2000, 2100, 2200, 2300, or 2400×g) for from about 15 mins to about 1 hour (e.g., about 20, 30, 40, or 50 mins.) Typically, CICs are incubated with from about 3 to about 3.5% PEG 8000 at 4° C. for about 20 h, and centrifuged at 1800 g for about 30 mins. CICs can be subsequently manipulated to separate the CICs into their various components, such as by separation of the components electrophoretically (e.g., on a denaturing or non-denaturing gel); denaturation using chaotropic or denaturant solutions; or separation on an affinity or sizing column (e.g., under denaturing or non-denaturing conditions).

In addition to the detection of enolase, including soluble enolase, the detection of enolase antibodies, or the detection of CICs that include enolase, the detection of viral FIP RNAs in a sample, including a sample that includes CICs, can provide confirmation of the exposure of the individual to FIP CoV or infection of the individual by FIP CoV. Methods known to those of ordinary skill in the art can be used to detect a viral FIP RNA, including Northern blots, Northwestern blots, PCR methods (including qualitative and quantitative PCR methods), RNA sequencing, or primer extension assays. Any region of a viral RNA, whether genomic or mRNA, can be detected, including untranslated regions (UTRs), such as a 3′UTR. In certain circumstances, a labeled polynucleotide probe, such as a labeled primer that is complementary to a viral RNA, can be used to detect the viral RNA, e.g., such as through hybridization. In other cases, a labeled polypeptide probe, such as a labeled enolase polypeptide, can be used to detect a viral RNA.

Methods for Screening for Multi-Organ Coronavirus Infection or Autoimmune Diseases Associated with Multi-Organ CoV Infection

The described methods can be used to screen for multi-organ coronavirus infection or exposure and/or autoimmune diseases associated with such an infection. While not being bound by theory, it is believed that infection with a multi-organ coronavirus, such as FIP, SARS, or a SARS-like virus, can result in an autoimmune pathology, leading to the accumulation of CICs that include enolase, the release of soluble enolase from targeted, lysed and/or apoptotic cells, and the production of anti-enolase antibodies. In this regard, it is noteworthy that anti-FIP CoV antibodies cross-react with SARS-CoVs in cell cultures (e.g., Ksiazek et al., “A novel coronavirus associated with Severe Acute Respiratory Syndrome,” New England J. Med. 348:1953-1966 (2003)). Also, as noted previously, experimental SARS and FIP CoV vaccines both resulted in a more fulminant pathology of the respective disease upon subsequent infection. Further, the spike proteins of SARS and FIP CoVs exhibit regions of nucleotide and amino acid identity (e.g., Stavrinides and Guttman, “Mosaic evolution of the severe acute respiratory syndrome coronavirus,” J. Virol. 78(1):76-82 (2004) and Example 5, below). Interestingly, both the SARS and FIP CoVs spike proteins also exhibit regions of moderate identity (about 20-50%) with human alpha-enolase. For example, a 16 amino acid sequence from the SARS spike protein shares 50% amino acid identity with a region of human alpha-enolase (Example 5, below). Thus, antigenic mimicry between regions of the spike proteins of SARS and FIP and host enolase could represent a possible mechanism for the triggering of an autoimmune response.

Accordingly, any of the described methods for screening an individual of the Felidae family for FIP CoV exposure or infection can be employed to screen an individual, e.g., a mammal, including individuals of the Felidae, Viverridae, or Mustelidae families, or a bird, for exposure to or infection by a multi-organ coronavirus or an autoimmune disease associated with such a virus. The method can include one or more of the following steps, either alone or in any combination or order:
(a) determining whether or not a sample that includes circulating immune complexes from the individual includes enolase;
(b) determining whether or not a sample (e.g., a biological fluid sample) from the individual includes soluble enolase;
(c) determining whether or not a sample from the individual, including a sample that contains CICs, includes antibodies specific for enolase; and/or
(d) determining whether or not a sample that includes circulating immune complexes from the individual includes multi-organ coronavirus RNA, including the 3′UTR.

Any of the methods described previously can be used in such screens, including sandwich and multi-layer assays. As it is believed that the pathophysiology of multi-organ coronaviruses is a result of an induced autoimmune response after exposure or infection, similar methods as outlined previously can also be used to determine if an individual that has been exposed to a multi-organ coronavirus is likely to develop a multi-organ pathology, including an autoimmune pathology, as a result of the exposure.

Methods for Evaluating Multi-Organ CoV Vaccines

As noted previously, experimental vaccines to SARS and FIP CoV have resulted in more fulminant forms of the diseases upon subsequent exposure to the viruses. Such an increase in severity can be due to antibody-dependent enhancement (ADE) of the disease, possibly as a result of the autoimmune pathophysiology described herein. Accordingly, it would be useful to screen candidate experimental vaccines for the treatment or prevention of a multi-organ coronavirus, such as FIP CoV, SARS CoV, a SARS-like virus, or newly-emerging systemic CoVs, to determine if a vaccine induces one or more of the biomarkers (e.g., CICs comprising enolase; soluble enolase; antibodies to enolase) linked to multi-organ coronavirus exposure or infection and associated autoimmune responses. For example, both existing and experimental vaccines can be evaluated. As used herein, the term “test vaccine” encompasses both existing and experimental vaccines. Thus, the invention provides a method for determining whether or not a test vaccine for a multi-organ CoV is safe for administration. The method includes:
(a) administering the test vaccine to an individual; and one or more of the following steps, in any combination or order:
determining whether or not an elevated level of antibodies specific for enolase is produced in the individual relative to a control individual not administered the test vaccine, wherein an elevated level of antibodies specific for enolase is indicative that the test vaccine is not safe for administration;
determining whether or not an elevated level of free enolase in serum or bodily fluids of the individual is produced relative to a control individual not administered the test vaccine, wherein an elevated level of free enolase is indicative that the test vaccine is not safe for administration; and
determining whether or not an elevated level of CICs comprising enolase are produced in the individual relative to a control individual not administered the test vaccine, wherein an elevated level of CICs comprising enolase is indicative that the test vaccine is not safe for administration.

Any of the methods described previously can be used, including sandwich and multi-layer assays. Similar methods can also be used to evaluate if a test vaccine has an increased tendency to induce an ADE response in a individual. In these cases, an elevated level (e.g., of CICs comprising enolase; of free enolase; or of antibodies to enolase) is indicative that a test vaccine has an increased tendency to induce an ADE response.

It should be noted that, with respect to FIP CoV test vaccines, a control individual can be an individual not administered the test vaccine, as indicated above, or can be an individual that has been vaccinated with Primucell™ vaccine. The inventors have discovered that Primucell™ vaccine does not result in elevated levels of the Averill biomarkers (elevated levels of free enolase, antibodies to enolase, or CICs that include enolase), and thus provides a useful control reference.

Methods for Screening Agents to Treat or Prevent Multi-Organ Coronavirus Infection or Autoimmune Diseases Associated with Multi-Organ CoV

The invention also provides methods for screening a test agent (e.g., a compound such as a small molecule organic compound, polypeptide, or polynucleotide) to determine if it is useful for preventing or treating a multi-organ CoV infection. The method takes advantage of the discovery that alpha-enolase binds to the 3′UTR of FIP RNA. Disruption of such binding by, e.g., small molecules, could provide a mechanism to inhibit the autoimmune cascade that manifests itself after exposure to or infection with a multi-organ CoV. The method includes:
(a) contacting a complex of enolase and multi-organ CoV RNA with a test agent; and
(b) determining if the test agent disrupts the complex.

Any method known to those having ordinary skill in the art can be used to evaluate disruption of the complex. For example, a variety of competitive assays using varying concentrations of a test agent can be employed to determine if a test agent competes with enolase for binding to a viral RNA. A viral RNA can include a 3′UTR and can be genomic or mRNA. In certain cases, viral FIP RNA can be employed, which can include the 3′UTR. In other cases, a 3′UTR of SARS CoV can be employed, or a 3′UTR of FIP CoV.

In certain cases, an aptamer of the 3′UTR that interacts with the epitope of enolase can be used. Such an aptamer can be determined by deletion analysis of the 3′UTR and/or deletion analysis/mutational analysis of enolase.

Methods for Distinguishing Protective Immunogenic Protein Domains from Auto-Immunogenic Protein Domains

As mentioned herein, both SARS and FIP CoVs spike proteins exhibit regions of moderate identity (about 20-50%) with human alpha-enolase. Thus, antigenic mimicry between regions of the spike proteins of SARS and FIP and one or more host enolase polypeptides could be a possible mechanism for triggering of an undesired autoimmune response, including an ADE response. It would be useful, therefore, to have a method that can distinguish a protective immunogenic viral polypeptide (e.g., an FIPV protein or a SARS protein) or a domain or fragment thereof, from a viral polypeptide, or a domain or fragment thereof, that induces auto-antibodies to an auto-polypeptide (or a domain or fragment thereof) in a mammal. As used herein, an auto-polypeptide is a polypeptide synthesized by an animal and not by a virus (e.g., FIPV or SARS). In addition, a “protective immunogenic polypeptide (or domain or region thereof)” is a polypeptide (or domain or fragment thereof) of a pathogen (e.g., a virus such as FIPV), which can induce antibodies that protect against the pathogen but that do not engender an undesirable autoimmune response, such as, but not limited to, the production of auto-antibodies to an autopolypeptide or the occurrence of an ADE response.

Accordingly, a method for distinguishing a protective immunogenic viral polypeptide from a viral polypeptide that induces auto-antibodies to an auto-polypeptide can include:
contacting a viral polypeptide (or a domain or fragment thereof) with one or more antibodies capable of recognizing, independently, one or more candidate auto-polypeptides (or a domain or fragment thereof); and
determining whether or not the one or more antibodies recognizes the viral polypeptide (or domain or fragment thereof).

A determination of recognition by one or more antibodies can be indicative that a viral polypeptide (or domain or fragment thereof) can induce the production of auto-antibodies to the auto-polypeptide in a mammal. For example, a determination that one or more antibodies which recognize alpha-enolase can also recognize FIP spike protein can be indicative that the inclusion of FIP spike protein in a vaccine could lead to the induction of auto-antibodies to alpha-enolase in a mammal administered the vaccine. The production of auto-antibodies can, in certain cases, be correlated with an increased tendency of the viral polypeptide (or domain or fragment thereof) to induce undesired autoimmune responses, including an ADE response, in a mammal.

It should be noted that similar methods can be used to distinguish protective immunogenic domains of an FIP polypeptide from domains of the same FIP polypeptide that induce the production of auto-antibodies to an auto-polypeptide. In such a method, one or more domains of an FIP viral polypeptide (e.g., a “candidate domain”) is contacted with one or more antibodies capable of recognizing one or more candidate autopolypeptides. Recognition of a candidate domain by one or more of the antibodies can be indicative that the candidate domain induces the production of auto-antibodies to the candidate auto-polypeptide in a mammal. Accordingly, such a candidate domain may be excluded from a vaccine preparation.

As used herein, a “fragment” is a portion or a region of a polypeptide. A fragment may encompass a few to many amino acids. For example, a fragment of a polypeptide can be 10 amino acids, 20, 30, 50, 100, 200, or >200 amino acids. In certain cases a “fragment” can be a domain of a polypeptide, e.g., a region of the polypeptide that is recognized by those having ordinary skill in the art to maintain certain structural and/or functional features (e.g., conserved domains). As used herein, a “domain” is any part of a polypeptide, which, when folded, creates its own hydrophobic core. A domain can act as independent unit, in the sense that it can be separated from a polypeptide chain, and still fold correctly, and often still exhibit biological activity.

Methods for the identification of a candidate auto-polypeptide (e.g., a candidate auto-polypeptide for screening for cross-reactivity with a viral polypeptide) are described herein (e.g., Example 1). Other methods also can be used to identify auto-polypeptides. For example, a candidate auto-polypeptide can be identified based on its status as a pathogen receptor, or if it is otherwise closely associated with a pathogen (e.g., through formation of a protein complex), by using immunological methods known to those having ordinary skill in the art. In some cases, a candidate auto-polypeptide can be identified if its coding sequence is highly expressed in infected individuals as compared to healthy individuals, using expression monitoring methods known to those having ordinary skill in the art.

Antibodies suitable for use in the method include, but are not limited to, unpurified antibodies (e.g., antibodies contained within a serum sample) or purified antibodies. Antibodies can recognize an intact auto-polypeptide, or a fragment or domain thereof. In certain cases, antibodies which recognize an auto-polypeptide can be commercially available. For example, Human Non-Neuronal Enolase (NNE)-rabbit polyclonal α-α-enolase antibody is available from Biogenesis (cat# 6880-0419). Human C-terminus of α-enolase/ENO1-goat polyclonal antibody is available from Santa Cruz Biotechnology (cat# sc-7455).

Antibodies that bind to an auto-polypeptide also can be produced by, for example, immunizing host animals (e.g., rabbits, chickens, mice, guinea pigs, or rats) with the auto-polypeptide. An auto-polypeptide or a fragment or domain thereof can be produced recombinantly, by chemical synthesis, or by purification of the native protein. An auto-polypeptide can then used to immunize animals by injection of the auto-polypeptide. Adjuvants can be used to increase the immunological response, depending on the host species. Suitable adjuvants include Freund's adjuvant (complete and incomplete), mineral gels such as aluminum hydroxide, surface active substances such as lysolecithin, pluronic polyols, polyanions, peptides, oil emulsions, keyhole limpet hemocyanin (KLH), and dinitrophenol. Standard techniques can be used to isolate antibodies generated in response to the auto-polypeptide immunogen from the sera of the host animals.

Methods which allow for the identification of antibody interactions with specific regions or domains of protein antigens can be referred to, in general, as epitope mapping. Inter alia, epitope mapping enables the determination of regions or domains of proteins that are likely to induce an immune response, including an undesired immune response. With such knowledge at hand, it is possible, for example, to design vaccines that contain protein epitopes which induce a protective immune response while minimizing the potential for an undesired immune response, such as an autoimmune response or an ADE response.

Methods of epitope mapping can be linear or conformational. Linear epitope identification determines an antibody binding site which is contained within a short and continuous secondary structure of a protein. Conformational epitope identification allows for identification of an antibody binding which is present on a tertiary, or native structure of a protein. Methods of linear epitope identification include, without limitation, Enzyme Linked Immuno Sorbent Assay (ELISA) and western blotting. Conformational epitope mapping methods include, for example, the use of phage-display libraries and protein chips. Epitope mapping services and kits are available from a number of vendors, including, New England BioLabs (Beverly, Mass.), Genencor (Palo Alto, Calif.), Applied Biosystems (Foster City, Calif.), and Pepscan Systems (Lelystad, The Netherlands).

Vaccines

It is contemplated that a vaccine for use in the treatment of mammals can be prepared with one or more coronavirus antigens (e.g., FIPV, SARS, and SARS-like coronavirus antigens). In some cases, the vaccine contains an immunogenic amount of one or more protective immunogenic FIPV antigens, e.g., a protective immunogenic FIP viral polypeptide or domain or fragment thereof determined as described above. A protective immunogenic FIPV antigen can stimulate the production of protective antibodies in mammals such as cats. Such FIPV antigens can be prepared, for example, by sub-cloning FIPV sequences of selected antigens or fragments thereof, and expressing such sequences using bacterial or mammalian expression systems. Such methods of sub-cloning and expression are known to those having ordinary skill in the art. Suitable FIPV antigens include, but are not limited to, FIPV nucleocapsid and spike polypeptides and domains or fragments thereof.

Certain vaccines described herein do not stimulate the production of auto-antibodies capable of recognizing (“specific for”) an enolase polypeptide of a host mammal. For example, a vaccine may not stimulate the production of auto-antibodies capable of recognizing an alpha-enolase polypeptide, or domains or fragments thereof. In certain cases, a vaccine may not stimulate the production of auto-antibodies capable of recognizing an amino-terminal domain of alpha-enolase (amino acids 1-300), or fragments of the amino-terminal domain. Thus, a vaccine may not stimulate the production of auto-antibodies capable of recognizing fragments containing amino acids 1-300, 1-250, 1-150, 1-100, 1-75, 1-50, 50-200, 75-250, 80-140, or amino acids 100-120 of a host's alpha-enolase.

A vaccine can be said “not to stimulate” or “not to induce” the production of auto-antibodies capable of recognizing an enolase polypeptide when a control mammal demonstrates levels of antibodies capable of recognizing the enolase polypeptide that are not statistically different from a time period before to a time period after administration of the vaccine. A control mammal can be any mammal as described previously. A time period can be, independently, any time period, e.g., 1 hr., 2 hr., 5 hr., 10 hr, 1 day, 2 days, 3 days, 5 days, 1 week, 2 weeks, 3 weeks, 4 weeks, etc.

Typically, a difference in levels of antibodies is considered statistically significant at p≦0.05 with an appropriate parametric or non-parametric statistic, e.g., Chi-square test, Student's t-test, Mann-Whitney test, or F-test. The absence of a statistically significant difference in, for example, the level of anti-enolase antibodies in a mammal after administration of a vaccine compared to the level in the mammal prior to administration of the vaccine indicates that (1) the vaccine does not induce auto-antibodies and/or (2) an FIPV antigen present in the vaccine warrants further study regarding the production of protective antibodies.

A vaccine typically is administered to a mammal in a physiologically acceptable, non-toxic vehicle, using, for example, effective amounts of immunological adjuvants. A virus antigen preparation can be conjugated or linked to a peptide or to a polysaccharide. For example, immunogenic proteins well known in the art, also known as “carriers,” may be employed. Useful immunogenic proteins include keyhole limpet hemocyanin (KLH), bovine serum albumin (BSA), ovalbumin, human serum albumin, human gamma globulin, chicken immunoglobulin G and bovine gamma globulin. Useful immunogenic polysaccharides include group A Streptococcal polysaccharide, C-polysaccharide from group B Streptococci, or the capsular polysaccharides of Streptococcus pnuemoniae or group B Streptococci. Alternatively, polysaccharides or proteins of other pathogens can be conjugated to, linked to, or mixed with the virus preparation.

A vaccine typically is administered to mammals parenterally, usually by intramuscular or subcutaneous injection in an appropriate vehicle. Other modes of administration, such as oral delivery, intranasal delivery, or mucosal delivery can also be suitable. In some cases, mammals can be administered vaccine compositions comprising a therapeutically effective amount of a polypeptide and/or polynucleotide, such as the soluble form of a polypeptide and/or polynucleotide, agonist or antagonist peptide or small molecule compound, in combination with an acceptable carrier or excipient. In some instances, mammals, such as cats, can be administered compositions comprising a therapeutically effective amount of a soluble form of one or more FIPV antigens, in combination with dextrose as a carrier.

Vaccine compositions can contain an effective amount of one or more FIPV antigens in a vehicle. An effective amount is sufficient to prevent, ameliorate or reduce the incidence of a viral infection (e.g., FIPV) in a target mammal, as determined by one skilled in the art. The amount of one or more FIPV antigens in a vaccine composition may range from about 1% to about 95% (w/w) of the composition. The quantity to be administered depends upon factors such as the age, sex, weight and physical condition of the mammal considered for vaccination. The quantity also depends upon the capacity of the mammal's immune system to synthesize antibodies, and the degree of protection desired. Effective dosages can be established by one of ordinary skill in the art through routine trials establishing dose response curves. Cats, for example, can be immunized by administration of the vaccine in one or more doses. Multiple doses may be administered if required to maintain a state of immunity to FIPV infection. The vaccine can be administered at different time intervals (e.g., daily, weekly, or even less often).

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid formulations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservatives.

To prepare a vaccine, antigens can be isolated, lyophilized and stabilized. The antigens may then be adjusted to an appropriate concentration, optionally combined with a suitable vaccine adjuvant, and packaged for use. Suitable adjuvants include but are not limited to surfactants, e.g., hexadecylamine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols; polyanions, e.g., pyran, dextran sulfate, poly IC, polyacrylic acid, carbopol; peptides, e.g., muramyl dipeptide, MPL, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). Finally, the immunogenic product may be incorporated into liposomes for use in a vaccine composition, or may be conjugated to proteins such as keyhole limpet hemocyanin (KLH) or human serum albumin (HSA) or other polymers.

Isolated Antibodies and Articles of Manufacture

The invention also features isolated antibodies and articles of manufacture for use in the described methods. For example, an article of manufacture described herein can be used to perform any of the methods described previously, e.g., sandwich or multi-layer assays to detect enolase or enolase antibodies.

An isolated antibody specific for enolase is provided. In certain cases, an isolated antibody specific for enolase can be specific for Felidae enolase (e.g., Felidae alpha-enolase) but not specific for human enolase (e.g., human alpha-enolase). In such cases, the isolated antibody specific for, e.g., Felidae alpha-enolase would not cross-react with, e.g., human alpha-enolase.

In other cases, an isolated monoclonal antibody can specifically bind to an FIPV epitope and not to enolase. For example, an isolated monoclonal antibody can specifically bind to the FIPV spike protein and not to alpha-enolase. In other cases, an isolated monoclonal antibody can specifically bind to the FIPV spike protein and not to a domain (e.g., the N-terminal domain or fragments thereof) of alpha-enolase.

An isolated antibody can be provided as part of an article of manufacture, such as a kit. Thus, an isolated antibody can be provided with packaging and a label providing instructions for use of the isolated antibody, such as for screening methods for multi-organ coronavirus exposure or infection.

An article of manufacture can, in certain circumstances, include a substrate, such as a solid substrate. A substrate can include a plurality of wells or defined regions, e.g., a microtiter plate, membrane, bead, particle, or array. A substrate can have immobilized thereon (either covalently or noncovalently) any of the Averill biomarkers described herein, e.g., one or more isoforms of enolase or an antibody specific for one or more isoforms of enolase, including an antibody specific for an isoform of Felidae enolase but not for the corresponding isoform of human enolase.

An article of manufacture can provide qualitative or quantitative measurements, and can be multiplex in nature, e.g., can screen for more than one biomarker of a multi-organ coronavirus infection and/or can screen for other diseases of interest. For example, with respect to cats, an article of manufacture can provide a panel screen for FIP CoV exposure or infection and for exposure to or infection with one or more of the following: Feline Herpes, Feline Calici, Feline Leukemia, Feline Immunodeficiency Virus, Feline Parvovirus, FECV, and vaccination for an FIP CoV. A panel screen can also screen for exposure to or infection to a variety of bacterial infections, Streptococcus sp., and Candida. A panel can be used to validate that a cat is pathogen-free with respect to certain pathogens, e.g., to validate use of a cat for cat models of human diseases. Thus, in certain cases, an article of manufacture can screen for any combination of Averill biomarkers, including soluble versions of alpha and gamma enolase and homo- and hetero-dimers of the same, and anti-enolase antibodies to alpha-enolase and gamma-enolase. An article of manufacture can further screen for biomarkers associated with other feline diseases, as discussed previously, and for FECV infection and/or FIP CoV vaccination (e.g., by detecting antibodies to the FIP spike protein, nucleoprotein, or whole virus).

Methods for Selecting Kittens for Breeding

The invention also provides a method for selecting an individual of the Felidae family for breeding or adoption (e.g., from a cattery, pet shop, shelter, or breeder). An individual of the Felidae family can be a kitten. The method includes determining one or more of the following:
(a) whether or not free enolase is present in the individual's serum or bodily fluids;
(b) whether or not antibodies to enolase are present in the individual's serum, bodily fluids, or in CICs; and/or
(c) whether or not CICs comprising enolase are present in the individual.

Any of the methods described previously can be used. A positive finding of (a), (b), or (c) is indicative that the individual has been exposed to FIP CoV and may be unsuitable for breeding or adoption, as the likelihood of the individual developing FIP-associated pathologies in the future is increased. The method can include determining one or more of (a), (b), or (c) over time. Monitoring over time can include determining the appropriate level at two or more time points, e.g., at two time points, for example, 1 month apart, 6 weeks apart, 2 months, 10 weeks apart, 3 months apart, 4 months apart, or 6 months apart.

### EXAMPLES

### Example 1

### Binding of 3′UTR(+Sense) of FIPV by Enolase

The 3αuntranslated region (UTR) of FIP was used as a riboprobe to determine if any host proteins bound to the viral FIP RNA. The 3′UTR was employed because this region is highly conserved among coronaviruses and is believed to be an important region involved in viral replication.

The interaction between the 3′UTR of FIP and host proteins was examined using one-dimensional sodium dodecyl sulfate polyacrylamide gel electrophoresis (SDS-PAGE) of host proteins followed by Northwestern analysis using a 3′UTR riboprobe of FIP. The interaction was further examined using a two-dimensional Northwestern assay probing with the 3′UTR FIPV riboprobe.

FIPV Virus Propagation

Crandell Feline Kidney (CrFK) cells were infected with DF2 strain (serotype II) of feline infectious peritonitis virus and incubated for one hour at 37° C. To virally infected flask, 10 ml of minimal essential media (MEM) supplemented with 7% fetal bovine serum (FBS) and 0.5% antibiotics were added. The flask was incubated overnight at 37° C. in 5% CO₂ incubator, and cytopathic effects were seen 20 hours post infection.

Total RNA Extraction from CrFK Flask Infected with FIPV

Supernatant from FIPV infected flask was removed and centrifuged at 3,000 rpm for 10 minutes to remove cellular debris and supernatant was collected. Viral RNA isolation was carried out using QIAampViral RNA Kit (Qiagen Inc., Valencia, Calif.) according to the manufacturer's instructions. FIPV RNA was absorbed onto a silica-gel membrane during two centrifugation steps; chaotropic salt and pH conditions in the lysate ensured that protein and other contaminants were not retained on the membrane. This viral RNA was eluted with high purity wash buffers and run on an agarose gel for size and purity determination.

Cloning of FIPV3′UTR in pGEM-T Prokaryotic Vector

PCR amplification of the FIPV 3′UTR gene was carried out with forward (5′-CAT CGC GCT GTC TAC TCT TG-3′; SEQ ID NO:1) and reverse (5′-TTG GCT CGT CAT AGC GGA TC-3′; SEQ ID NO:2) primers, which were designed based upon the feline coronavirus mRNA for 7a and 7b protein (strain FIPV Dalberg) gene sequence deposited in GenBank (Accession number X90572). The 3′UTR is common in all mRNAs and genomic RNA, thus providing enhanced sensitivity over other FIP RNA sequences. Total CrFK RNA from FIPV infected cell culture flask was used as a template for 100 μl reverse transcriptase polymerase chain reaction (RT-PCR) using GeneAmp RNA PCR kit from Perkin Elmer (Applied Biosystems, Foster City, Calif.). The conditions for reverse transcription were as follows: 42° C. for 30 minutes, 99° C. for 5 minutes, and 5° C. for 5 minutes and amplification, initial denaturation, 94° C. for 20° C. sec; 50° C. for 20 sec; 72° C. for 20 sec; 50 cycles of denaturation 50° C. for 20 sec 72° C. for 20 sec, and a final extension at 72° C. for 10 minutes. Amplified product was used in overnight 4° C. water bath in T4 DNA ligase reaction with pGEM-T prokaryotic expression vector (Promega, Madison, Wis.). Ligated DNA was electroporated with JM109 competent E. coli cells and plated on LB agar plus carbenicillin (20 mg/ml) with X-Gal (50 mg/ml) and 100 mM IPTG. Plates were placed at 37° C. overnight. Blue/White screening was used on bacterial colonies. White colonies were further plated on LB agar plus carbenicillin for growth overnight. Further in-well lysis colony screening was performed for positive colonies (the plasmid DNA which migrated higher than negative control blue colonies being selected). Alkaline lysis followed by polyethylene glycol (PEG 8000) precipitation was performed on suspected positive plasmid cultures. Verification of FIPV 3′UTR insert was performed by manual sequencing of purified DNA using SequiTherm EXCEL II DNA Sequencing Kit (Epicentre Technologies, Madison, Wis.) according to the manufacturer's instructions. A 122 bp insert of FIPV 3′UTR DNA was verified upon BLAST search of the sequence.

In Vitro Transcription

DNA from FIPV 3′UTR expression plasmid was purified and linearized with SacI enzyme. In the transcription reaction, 1.0 μg of linearized DNA was combined with 5×transcription buffer, 100 mM DTT, 10 mM ATP, 10 mM CTP, 10 mM UTP, 100 μM UTP, α-[P³²]-CTP, and T7 RNA polymerase enzyme according to the manufacturer's instructions (RiboScribe RNA Probe Synthesis Kit, Epicentre Technologies, Madison, Wis.). Using T7 RNA polymerase, the positive strand of FIPV 3′UTR RNA was produced. Transcriptionally unincorporated dNTPs were removed by use of G25 Sephadex columns (Quick Spin Columns (TE), Boehringer Mannheim, Indianapolis, Ind.).

Northwestern Blotting

Nitrocellulose membranes (one dimensional feline tissue and cytoplasmic cell lysates, or two dimensional electrophoretically separated CrFK proteins) were washed with constant shaking in 6M GnHCl for 1 hour. The proteins were renatured slowly by washing the blots with RNA binding buffer (0.05 M NaCl, 10 mM Tris pH 7, 1 mM EDTA, 0.02% Polyvinyl pyrrolidone, 0.02% bovine serum albumin, 0.02% Ficoll) every 10 minutes followed by a final wash of 45 minutes. The blots were incubated further with binding buffer containing 100 μg/ml salmon sperm DNA and 10 μg/ml of yeast tRNA for 1 hour to block nonspecific binding. Purified FIPV 3′UTR RNA labeled with α-P³² (at a concentration of 500,000 cpm/ml of binding buffer) was added and allowed to hybridize for 10 hours with gentle rotation. Unbound probe was removed by washing with binding buffer for 2 hours at 30 minute intervals, dried, and bound RNA was visualized by autoradiography at −80° C.

Isolation and Solubilization of Feline Tissue for One Dimensional SDS-PAGE

Feline tissues were obtained from cases submitted for post-mortem examination to Kansas State University College of Veterinary Medicine/Department of Diagnostic Medicine Pathobiology. Feline tissues were finely powdered after quick freezing in liquid nitrogen using the MIKRO-Dismembrator (B. Braun BioTech Inc, Allentown, Pa.) and liquid nitrogen. A suspension of tissue proteins was made in 0.01M PBS and frozen at −80° C. until further electophoresis and Northwestern blot analysis using FIPV 3′UTR RNA.

The effect of monovalent and divalent cations on feline protein-FIPV 3′UTR RNA binding was examined by using differing concentrations of KCl and NaCl on host protein-viral RNA complex formation through addition of ions to RNA binding buffer during northwestern hybridization. Enolase has a metal ion requirement for certain divalent metal ions for its activity. Magnesium is the natural cofactor.

Isolation and Solubilization of CrFK Proteins for Two Dimensional PAGE

Protein isolation was adapted from Rabilloud, “Proteome Research: Two-Dimensional Gel Electrophoresis and Identification Methods,” Springer, N.Y. (2000) for animal cell protein solubilization. Monolayered CrFK cells in 75 cm² flasks were washed once with 0.01M phosphate buffered saline followed by suspension in 10 mM Tris pH 7.5, 1 mM ethylene diamine tetraacetic acid (EDTA), 0.25 M sucrose buffer. One volume of suspension was placed in a polyallomer ultracentrifuge tube and four volumes of concentrated extraction buffer (9.6 M urea, 25 mM spermine tetrahydrochloride, 50 mM DTT) was added. Extraction was carried out at room temperature for one hour. Samples were ultracentrifuged at 250,000 g for 1 hour. A translucent pellet of nucleic acids was obtained. The protein containing supernatant was precipitated overnight in 75% acetone, 10% trichloroacetic acid at 4° C. Protein was then centrifuged 10,000 g for 30 minutes and the pellet resuspended in extraction buffer. Protein concentration was measured using BioRad's (Hercules, Calif.) microplate assay based on Bradford's method.

Two-Dimensional PAGE of CrFK Proteins

First dimension:Isoelectric Focusing (IEF)—Isolated CrFK proteins were further solubilized by resuspending 125 μg of protein in extraction buffer (9.6 M urea, 25 mM spermine tetrahydrochloride, 50 mM DTT) and Destreak Solution (Amersham Biosciences, Piscataway, N.J.) supplemented with ampholytes, pH 3.5-10 (Pharmalyte, Amersham Biosciences).

Immobilized pH gradient strips (BioRad), non-linear pH 3-10, 11 cm long and pH 5-8, 11 cm long were passively rehydrated overnight at room temperature. Strips were focused with maximal 8,000 V and 50 μA to reach 30,000 V-hr. After completion of focusing, strips were preserved at −80° C. until second dimension PAGE was performed.

Second dimension:PAGE—IEF strips were incubated in equilibration buffer I (6M urea, 2% SDS, 0.05M Tris-HCl pH 8.8, 20% gylcerol, 2% DTT) for ten minutes with gentle shaking followed by equilibration buffer II (6M urea, 2% SDS, 0.05M Tris-HCl, 20% glycerol, 2.5% iodoacetamide). The IEF strips were loaded onto pre-cast 10% Tris, 1.0 mm Criterion gels (BioRad), overlayed with 0.05% agarose with 0.002% bromophenol blue, and run at 150V for 1 hour. Parallel gels (identically rehydrated and electrophoresed) were either stained using BioRad's Silver Stain Plus Kit or transfer was made onto nitrocellulose membrane (Pall Gellman, Ann Arbor, Mich.) for northwestern blotting with FIPV 3′UTR RNA probe.

Analysis and Isolation of Proteins Binding to FIPV 3′UTR

During northwestern analysis, the film was carefully marked to outline each FIPV 3′UTR RNA probed nitrocellulose membrane before removal from autoradiography cassette. Blots were removed and staining according to Harlow and Lane, “Using Antibodies: A Laboratory Manual,” Cold Spring Harbor Laboratory Press, New York (1999), p. 295. Membranes were washed in buffer containing 0.3% Tween 20 in 0.01M phosphate buffered saline (PBS). Each blot was then placed in a 1.0% India Ink suspension in wash buffer and incubated at room temperature with gentle shaking for 10 hours and protein spots were visibly clear. Identical India Ink stained membrane was then aligned with protein-RNA signal on autoradiography film and matched with identically run silver stained gel. These were all aligned and distances between protein spots were calculated to locate RNA binding protein within silver stained gel for excision. A positively identified protein spot was excised from silver stained gel and placed in 0.5 ml microcentifuge tube for further mass spectroscopic analysis.

Results:

One Dimensional SDS-PAGE/North Western Blot of CrFk Proteins

Nitrocellulose membranes of proteins were denatured in 6M guanidinium hydrochloride for 30 minutes followed by prehybridization in SBB buffer containing 0.05M NaCl, 10 mM Tris pH 7.0, 1 mM EDTA, 0.02% BSA, 0.02% Ficoll, 0.02% polyvinyl pyrrolidone. Hybridization with ³²P-labeled 3′UTR FIPV RNA probe at 500,000 cpm/ml along with 10 μg/ml of tRNA and 100 μg/ml of sheared salmon sperm DNA was made overnight. Blots were washed with SBB buffer for 1 hour 30 minutes and placed in intensifying autoradiography cassettes.

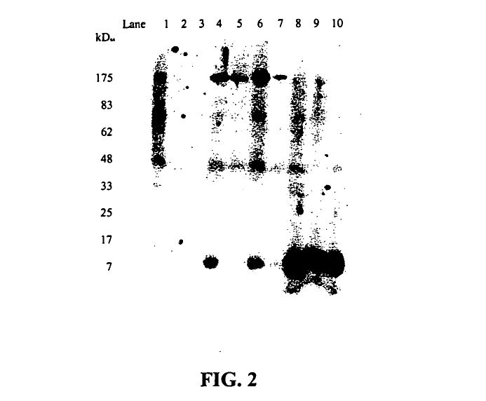

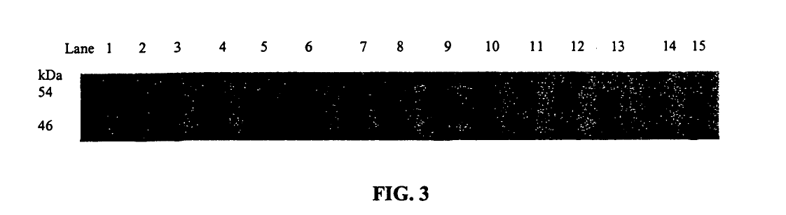

Five bands were detected in susceptible cell lines and tissues from cats; See FIG. 2. The 48 kDa band was later identified as alpha enolase by protein sequencing.

Two Dimensional Electrophoresis/North Western Blot

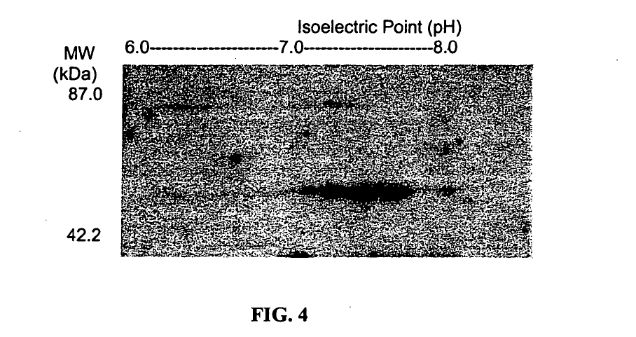

Proteins from a susceptible cell line, Crandel Feline Kidney Cell Line, were resolved by two dimensional electrophoresis. The first dimension allowed the separation of the proteins based on isoelectric point and second dimension allowed separation based on molecular mass. The protein spots were found to be reproducible and detectable by silver staining. After performing the 2D electrophoresis in a reproducible manner, the 2D separated proteins were transferred to a nitrocellulose membrane and probed with FIPV 3′ UTR riboprobe. The alignment of RNA-binding proteins with the silver stained gels was confirmed by Ink staining of the membranes. One pattern of these spots was seen repeatedly on two dimensional northwestern assay. See FIG. 4.

After MALDI-MS sequencing of the putative RNA-binding protein, it was identified as alpha enolase (see below).

Expressional Differences in Wet and Dry Forms of FIP

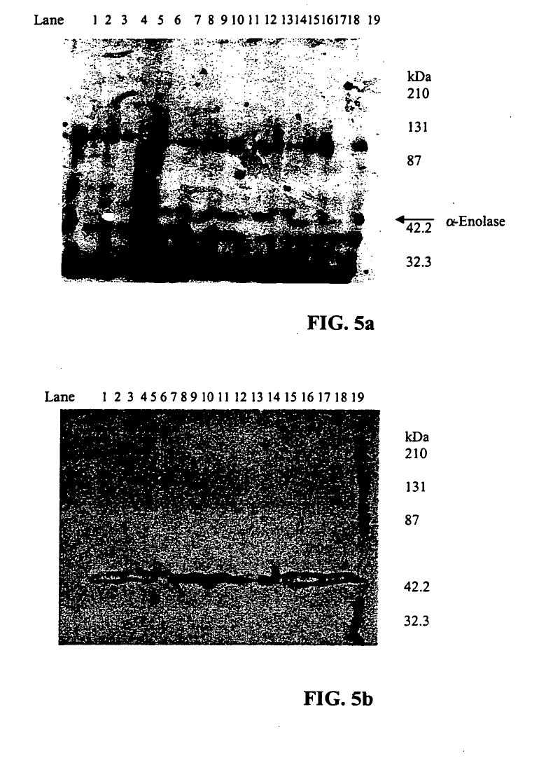

Tissues from cats with wet (effusive) and dry (non-effusive) forms of FIP were homogenized and the total proteins from tissues were electrophoresed on SDS-PAGE gels. After transfer to nitrocellulose membrane, the membranes were hybridized with the FIPV 3′ UTR riboprobe. After autoradiography, the molecular mass of the RNA-binding proteins were recorded. Alpha-enolase was detected in all feline tissues tested using goat anti-human alpha enolase antibody (Santa Cruz Biotechnology); however, differential binding of the 3′UTR FIPV RNA in feline tissues was seen between the wet and dry forms of the disease. See FIG. 5.

### Example 2

### Identification of Enolase as a Protein that Binds the 3′UTR of FIP RNA

Matrix Assisted Laser Desorption Ionization Mass Spectrometry (MALDI-MS)

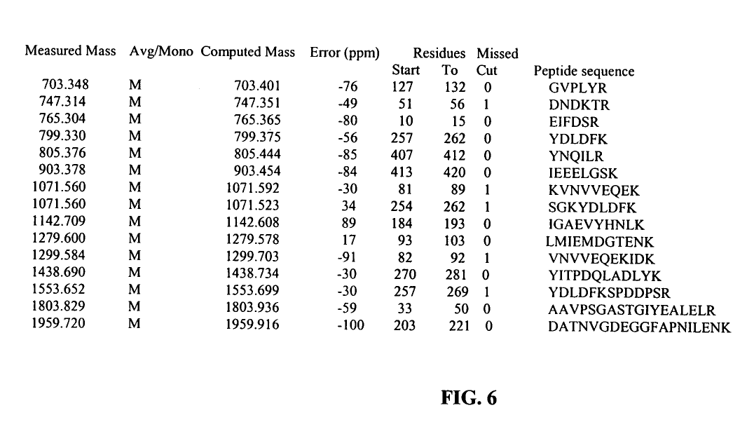

Protein spots were excised and sent to the University of Louisville Mass Spectroscopy Core Laboratory and the Yale Cancer Center Mass Spectrometry Resource/HHMI Biopolymer Laboratory for protein mass analysis. Briefly, a 1.5 ml tube was washed with 500 μl 0.1% TFA/60% CH₃CN, vortexed, and wash removed. The excised gel piece was placed in a prewashed tube and controls were placed in separate prewashed tubes (a transferrin control containing 10 pmol, and a blank piece of gel that did not contain protein). To each tube, 250 μl of 50% CH₃CN/50 mM NH₄HCO₃ was added. Samples were washed at room temperature for 30 minutes with gentle tilting. Wash was removed and 2501 μl of 50% CH₃CN/10 mM NH₄HCO₃ was added to gel pieces. Washing was done for 30 minutes at room temperature and wash removed. Gel pieces were placed in SPEEDVAC to complete dryness and trypsin (10 μl of 0.1 mg/ml trypsin stock diluted with 140 μl 10 mM NH₄HCO₃) added of equal volume to samples and blank. Additional 10 mM NH₄HCO₃ was added to tubes to cover the gel pieces and samples were incubated at 37° C. for 18 hours. Mass spectral data were obtained on a 5% aliquot of the digest using a Micromass Tof-Spec 2E instrument. Programs used for database searching included ProFound, which relies on the NCBI non redundant database, and the Mascot algorithm, according to standard protocos. The criteria used for identifying the protein included matching of peptide masses to >25% of the predicted protein sequence using a mass tolerance of ±0.0007% (70 ppm) for monoisotopic masses, a ProFound score of 1.0 with a clear break between this score and the score of the next, non-related protein. ProFound search is carried out with a mass range that extends from 50% to 150% of the MW estimated from SDS PAGE and without specifying taxonomic category. Mascot identifies the same proteins(s) as evidenced by a clear break in the number of peptides matched between the identified and the next highest ranked protein. A top score obtained was 1.0e+00 to alpha-enolase. The % coverage of the known sequence for this protein was 29%. A second Profound search was performed (after deleting masses which matched) with no additional protein being identified. Mascot matched the same protein with a clear break. Since all three of the criteria were met, this data demonstrated that the detected protein was alpha-enolase. See FIG. 6.

### Example 3

### Characterization of FIPV Immune Complexes: Demonstration of the Presence of Enolase in CICs

Given the binding of alpha enolase to the 3′UTR, the role of enolase in the immunopathology of FIPV was investigated. The pathophysiology of feline infectious peritonitis is associated with the formation of circulating immune complexes (CICs) that are deposited in various tissues and organs of affected cats. A better understanding of the pathogenesis of FIP was facilitated by identification of the components within the CICs.

Isolation of Feline Circulating Immune Complex (CIC)

Polyethylene glycol (PEG) precipitation of immune complexes was performed on feline sera and peritoneal fluid taken from cases submitted to the Kansas State University College of Veterinary Medicine Diagnostic Laboratory and confirmed to have feline infectious peritonitis by histopathology. Precipitation of immune complexes was a modification of L. Kestens et al., “HIV antigen detection in circulating immune complexes,” J. Virol. Methods 31:67-76 (1991) and L. Bode et al., “Boma disease virus-specific circulating immune complexes, antigenemia, and free antibodies—the key market triplet determining infection and prevailing in severe mood disorders,” Molecular Psychiatry 6:481-491 (2001). To one milliliter of serum, 5 ml of 3% PEG 8000 in 0.01M PBS was added and incubated for 20 h at 4° C. with gentle rotation. The precipitate was centrifuged at 1800 g for 30 minutes and the pellet resuspended in 1 ml 0.01 M PBS after which 200 μl 1M glycine-HCl (pH 2) containing 0.25% sodium dodecyl sulfate (SDS) was added. The sample was heated for 10 minutes at 70° C. followed by neutralization with 150 μl 1M Tris base. Samples were stored at −80° C. until further use.

Characterization of Alpha Enolase in FIPV Immune Complex

Approximately 10 μg precipitated immune complex were run on a 10% denaturing SDS-PAGE gel and blotted onto nitrocellulose membrane (Pall Gellman, Ann Arbor, Mich.). The immune complex blot was washed twice for 5 minutes with deionized water and placed in blocking solution (5% skim milk, 0.05% Tween 20, and 0.01M PBS) for 1 hour. Affinity-purified goat polyclonal IgG antibody to human enolase (Santa Cruz Biotechnology, Santa Cruz, Calif.) was then added to the blocking solution at a concentration of 1:1000 and the blot incubated at 4° C. overnight. This antibody was recommended by the manufacturer for the detection of alpha, beta, and gamma enolase of mouse, rabbit, human, and yeast. The blot was then washed twice for 5 minutes with 0.05% Tween 20/0.01M PBS. Peroxidase labeled horse anti-Goat IgG (H+L) (Vector Laboratories, Burlingame, Calif.) was added at 1:50 dilution in blocking solution and incubated for 2 hours at 4° C. After two washes in 0.05% Tween/0.01M PBS, TMB membrane substrate (Kirkegaard and Perry Laboratories, Gaithersburg, Md.[3,3′,5,5′-Tetramethylbenzidine]) was added. Single bands of approximately 47 kDa were observed in the gel, indicating the presence of enolase in circulating immune complexes.

### Example 4

### Demonstration of the Presence of Antibodies to Enolase in Serum and Tissues

Indirect Enzyme Linked Immunosorbent Assay (ELISA) for Detection of Alpha Enolase and Neuron Specific Enolase (NSE) Antibodies in Feline Sera

FIPV challenged feline sera and feline sera, including sera from both large (non-domestic) and small cats and from vaccinated cats, were used for testing for the presence of antibodies to alpha enolase and neuron specific enolase (NSE, which includes a mixture of a gamma-gamma homodimer and the alpha-gamma heterodimer). Fifty microliters of purified alpha enolase from yeast (ICN Biomedicals, Aurora, Ohio) was coated onto Immulon I flat bottom 96-well plates (Dynatech Laboratories, Chantilly, Va.) at 2 μg/ml diluted in coating buffer (45 mM NaHCO₃ and 182 mM Na₂CO₃ in deionized water pH 9.55) and incubated at room temperature for 2 hours. The plate was washed with buffer containing 0.01M PBS/0.05% Tween 20 and 100 μl of blocking solution (5% skim milk [Difco, Detroit, Mich.] in wash buffer) was added for 30 minutes at 37° C. After the plate was washed three times, 50 μl of feline serum diluted 1:50 in blocking solution was added to wells in triplicate and the plate was placed at 37° C. for 30 minutes. Following incubation, 50 μl of 1:10,000 dilution of secondary peroxidase labeled goat anti-cat IgG [H+L] antibody (Kirkegaard and Perry Laboratories, Gaithersburg, Md.) diluted in 5% skim milk was added to plate and incubated at 37° C. for 30 minutes. Secondary antibody was washed from plate and 50 μl substrate reagent (TMB Microwell [3,3′,5,5′-tetramethylbenzidine], Kirkegaard and Perry Laboratories, Gaithersburg, Md.) was added to each well and allowed to proceed at room temperature for 30 minutes before 50 μl of stop solution (1N H₂SO₄) was added. Absorbance was measured on a microtiter spectrophotometer at 450 nm.

The ELISA procedure was also performed with the substitution of purified neuron specific enolase from human brain (SIGMA-ALDRICH, St. Louis, Mo.) at 2 μg/ml diluted in coating buffer (45 mM NaHCO₃ and 182 mM Na₂CO₃ in deionized water pH 9.55).

Results: Antibodies to alpha enolase were not detected in uninfected, unvaccinated cats. Both domestic and large cats (including leopards, cougars, tigers, white tigers, snow leopards, bobcats, lions, pallas cats, and pumas) that had been exposed to FIP or were suspected of having been exposed to FIP demonstrated higher levels of antibodies to alpha enolase and/or NSE than healthy cats. Cats that had been vaccinated to FIP with Primucell™ had undetectable levels of antibodies to either alpha or NSE after challenge with FIP. The results indicate that the detection of antibodies to isoforms of enolase indicates exposure to the virulent FIP CoV.

Four samples from one household having four cats where one of the cat had died of FIP were also examined. The three living cats had come into contact with the FIP-CoV infected cat and were positive for alpha-enolase antibodies. Over the course of the following several weeks, alpha enolase antibody titers increased dramatically in the 3 living cats.

Quantitative EIA for Detection of Neuron Specific Enolase (NSE) in Feline Serum (Free or Soluble Enolase)

Feline serum samples obtained from FIPV challenge studies were used in a commercial neuron specific enolase EIA assay performed according to the manufacturer's instructions (ALPCO Diagnostics, Windham, N.H.). This Neuron Specific (NSE) EIA is a solid phase, non-competitive assay based on two monoclonal antibodies (derived form mice) directed against two separate antigenic determinants of the NSE molecule. The monoclonal antibodies used bind to the γ-subunit of the enzyme, and thereby detect both the homodimeric γγ and the heterodimeric αγ forms of enolase. NSE levels are low (15 ug/L or less) in healthy human subjects and subjects with benign diseases. Elevated NSE levels are commonly found in patients with malignant tumors and neuroendocrine differentiation, especially small lung cell lung cancer (SCLC) and neuroblastoma.

Standards and sera samples were incubated together with biotinylated anti-NSE monoclonal antibody E21 and horseradish peroxidase labeled anti-NSE monoclonal antibody E17 in streptavidin coated microtiter strips with gentle shaking. After washing, buffered substrate/chromagen reagent (hydrogen peroxide and 3,3′,5,5′-tetramethylbenzidine) was added to each well and the enzyme reaction was allowed to proceed. During the enzyme reaction a blue color developed if a positive antigen reaction was present. Absorbance was determined in a microtiter spectrophotometer at 405 after stop solution was added. Standard curves were constructed for each assay by plotting absorbance value versus the concentration of standards. The NSE concentrations of patient samples were then calculated from the standard curve.

Results: Cats exposed to FIP exhibited increased levels of free neuron specific enolase in sera and/or ascites fluid as compared to isolated or healthy cats.

Immunohistochemistry of Feline Tissues for Detection of Alpha Enolase

Fresh feline tissue sections were snap frozen and cut onto pretreated slides. The staining protocol was according to the goat ABC staining system (Santa Cruz Biotechnology, Santa Cruz, Calif.). Slides were fixed in cold acetone (−20° C.) for 15 minutes and incubated with 0.5% hydrogen peroxide in 0.01M PBS to reduce endogenous peroxide activity in feline tissues. After washing slides twice for 5 minutes each in 0.01 M PBS, slides were incubated 1 hour in 1.5% donkey serum in 0.01 M PBS to block unoccupied sites to prevent nonspecific binding. 5.0 μg/ml of primary goat anti-human alpha enolase antibody (Santa Cruz Biotechnology, Santa Cruz, Calif.) was then added and slides were incubated at 4° C. overnight. Three changes of 0.01M PBS for 5 minutes each was made and slides were incubated for 30 minutes with biotin-conjugated secondary antibody (1 μg/ml diluted in 1.5% donkey serum). Slides were washed in 3 changes of 0.01M PBS for 5 minutes each and slides were incubated in peroxidase substrate with DAB chromagen for 7 minutes. Sections were washed in deionized water for 5 minutes, counterstained with hematoxylin for 30 seconds, and washed with deionized water. Slides were dipped in 100% ethanol for 20 seconds followed by dipping for 10 seconds in xylene. Mounting media was added coversliped and slides visualized by light microscopy.

Results: Enolase was detected along the basement membrane of glomeruli in the kidney of a cat that had died of FIP. As CICs are deposited in basement membranes in the pathophysiology of FIP, and as CICs have been demonstrated to include enolase herein, the finding confirms the FIP diagnosis.

### Example 5

### Antigenic Mimicry Between Enolase and the FIP and SARS Spike Proteins

Virus Purification

CrFK cells were infected with FIPV DF2 and harvested when 75% of cells showed cytopathic effect, approximately 46 hours post infection. Flasks were freeze-thawed three times and cells were scraped and pooled. Cellular debris was removed by low speed centrifugation at 1,000 g for 15 minutes at 4° C., and the supernatants were precipitated with 8% (w/v) polyethylene glycol (PEG 8000). After 24 hour incubation at 4° C., the virus was pelleted at 9,000 rpm for 20 minutes. The pellet was resuspended in TNE buffer pH 7.5 (100 mM NaCl, 10 mM Tris/HCl pH 8.0, 1 mM EDTA), placed on continuous sucrose gradients of 20 to 60% w/w in TNE buffer, and ultracentrifuged at 90,000 g for 14 hours at 4° C. Following centrifugation, fractions were collected, diluted in TNE buffer and pelleted by centrifugation at 90,000 g for 2 hours at 4° C. Purified FIPV virions were saved at −20° C. until further analysis.

Identification of FIPV Spike Protein-Human Alpha Enolase Antibody Binding

Purified FIPV was run on a 10% denaturing SDS-PAGE gel and transferred to nitrocellulose membrane (Pall Gellman, Ann Arbor, Mich.). Blocking solution (5% skim, milk in 0.01M PBS) was added to membrane and the membrane was incubated at 4° C. for 1 hour with gentle shaking. Added to separate strips with dilution made in blocking solution were feline infectious peritonitis virus type 1 antiserum (VMRD, Inc. Pullman, Wash.) at 1:1000, feline infectious peritonitis virus type 2 antiserum (VMRD, Inc. Pullman, Wash.) at 1:1000, and polyclonal goat anti-human enolase antibody (Santa Cruz Biotechnology, Santa Cruz, Calif.) at a dilution of 1:500. Blots were incubated with primary antibody overnight at 4° C. Three washes were made with 0.01M PBS/0.05% Tween 20 and goat anti-cat IgG [H+L] antibody (Kirkegaard and Perry Laboratories, Gaithersburg, Md.) at a dilution of 1:10,000 was added to FIPV antiserum blots while horse anti-goat IgG (H+L) (Vector, Burlingame, Calif.) at a dilution of 1:10,000 was added to the enolase blot. After incubation at 4° C. for 2 hours, blots were washed twice and substrate (TMB membrane, Kirkegaard and Perry Laboratories, Gaithersburg, Md.) added for visualization to presence of enolase. A protein band of approximately 200 kDa, known to correspond to the spike protein of FIPV, was seen in the FIP-1 antiserum blot and aligned with a similarly sized protein band in the anti-human alpha enolase blot. Cross-reactivity of the enolase antibody with an FIPV protein demonstrates epitope similarity between regions of alpha enolase and the FIPV spike protein. A lower molecular weight protein of approximately 46 was seen in the FIP-2 antiserum blot which indicates this antiserum as recognizing the nucleocapsid (N) protein of FIPV.

Amino Acid Sequence Alignments

Amino acid sequence alignments between the spike proteins of FIP and SARS and human enolase suggested the presence of antigenic mimicry between fragments or domains of enolase and the two CoV spike proteins, perhaps indicating a mechanism for the induction of an autoimmune response for both CoVs in infected individuals. The LALIGN program was used for alignment with default parameters (see Huang and Miller, Adv. Appl. Math (1991) 12:337-357).

Moderate levels of identity (about 20-50%) were seen between human enolase isoforms and the SARS spike protein. Similar levels of identity were seen between the FIP spike protein and the human enolase isoforms.

A short region of 16 amino acids from SARS and human enolase alpha share 50% identity:
ILPDPLKPTKRSFIED (SEQ ID NO:5) from SARS spike protein with ISPDQLADLYKFIKD (SEQ ID NO:6) from human alpha enolase.

The SARS spike protein may express SEQ ID NO:5 as two antigenic peptide regions:
residue 783 NFSQILPDPLK 793 (SEQ ID NO:7) and residue 799 FIEDLLFNKVTLAD 812 (SEQ ID NO:8). The prediction of antigenic peptides was performed using the program available at http://mif.dfci.Harvard.edu/tools/antigenic.pl.

### Example 6

### Quantification of Soluble Enolase by ELISA as a Measure of Damage to Macrophages by FIP CoV

Macrophages are the principal cell types infected by CoVs. Once infected, as shown herein, macrophages release enolase, triggering the induction of the autoimmune response; such a response can be dependent on the genetic background and housing conditions of the individual (e.g., a cat). The induction of antibodies may happen because of the binding of FIP 3′UTR to alpha enolase to expose the cryptic antigenic domains of the enolase protein, rendering them immunogenic. As antibody titers can be dependent on the time of exposure of the affected cat, they can vary significantly. Direct quantification of enolase release can provide a more useful indication of damage to macrophages.

ELISA plates will be coated with anti-enolase antibodies in carbonate buffer. Sera from a cat suspected of having FIP will be added and allowed to incubate at 37° C., followed by washing. Anti-enolase second-site antibody labeled with horseradish peroxidase will be added, and color developed with soluble TMB. The amount of enolase detected will be a direct measure of FIP damage to the cat and a predictor of the outcome of the disease.

### Example 7

### Reactivity and Specificity of Enolase and FIP CoV Antibodies

Western Blot Analyses with Anti-Enolase and Anti-FIP Sera

Western blot analyses were carried out to test the specificity and reactivity of anti-enolase and anti-FIPV antibodies. Purified FIPV, purified alpha-enolase (Research Diagnostics, Flanders, N.J., RDI-TRK8NS4), purified beta-enolase (Sigma-Aldrich, St. Louis, Mo., E0379), and purified gamma-enolase (Research Diagnostics, RDI-TRK8NS3), respectively, were loaded and run on 10% denaturing SDS-PAGE gels and transferred to nitrocellulose membranes (Pall Gellman, Ann Arbor, Mich.). The membranes were next placed at 4° C. for 30 min and gently shaken in the presence of a blocking buffer (5% skim milk in 0.01M PBS with 0.05% Tween-20). Blot lanes of each antigen were separated and subjected to incubation as follows.

In order to determine the reactivity and specificity of sera which recognize FIP, blots were incubated with either FIPV positive serum from KSU case number 9190-2 at a dilution of 1:50. Feline infectious peritonitis virus type 1 antiserum at 1:1000 dilution (VMRD, Inc. Pullman, Wash., 210-70-FIPI), Feline infectious peritonitis virus type 2 antiserum at a dilution of 1:1,000 (VMRD, Inc. Pullman, Wash., 210-70-FIP2), or mouse monoclonal reactive with types 1 and 2 of feline infectious peritonitis virus (Serotec, Raleigh, N.C., MCA2194) at a dilution of 1:500. To determine the reactivity and specificity of sera which recognize alpha enolase, blots were incubated with polyclonal rabbit anti-human alpha-enolase amino-terminus serum (residues 1-300) at a dilution of 1:500 (Santa Cruz Biotechnology, Santa Cruz, Calif., sc-15343), polyclonal goat anti-alpha enolase carboxy-terminus serum (C-19) at a dilution of 1:500 (Santa Cruz Biotechnology, sc-7455), or polyclonal rabbit anti-human alpha-alpha enolase serum at a dilution of 1:500 (Biogenesis, Kingston, N.H., 6880-0410).

In order to determine the reactivity and specificity of sera which recognize beta-enolase, blots were incubated with mouse anti-beta enolase serum at a dilution of 1:500 (BD Transduction Laboratories, San Jose, Calif., E84420). In order to determine the reactivity and specificity of sera which recognizes gamma-enolase, blots were incubated with either mouse monoclonal gamma enolase antibody (residues 416-433) at a dilution of 1:500 (Santa Cruz Biotechnology, sc-21738), or mouse anti-gamma enolase IgG1 (residuels 271-285) at a dilution of 1:500 (Santa Cruz Biotechnology, sc-21737). To determine the reactivity and specificity of a serum which recognizes a virus other than FIP, blots were incubated with Transmissible Gastroenteritis Virus (TGEV) polyclonal serum at a dilution of 1:250 (National Veterinary Services Laboratories, 325PDV.

Following an overnight incubation with the respective primary antibodies at 4° C., blots were washed three times with 0.01M PBS/0.05% Tween-20. Goat anti-cat IgG HRPO-labeled antibody at a dilution of 1:10,000 (Kirkegaard and Perry Laboratories, Gaithersburg, Md.) was added to blots containing FIPV, while HRPO-horse anti-goat IgG at a dilution of 1:10,000 (Vector, Burlingame, Calif.) was added to blots containing alpha-, beta-, or gamma-enolase. Following incubation at 4° C. for 2 h, the blots were washed three times and then developed with 3,3′,5,5′-tetramethylbenzidine (THB membrane, Kirkegaard and Perry Laboratories, Gaithersburg, Md.). Table 1 summarizes the results of the western blot analyses.

TABLE 1

| | PROTEIN | | | | | |
|---|---|---|---|---|---|---|
| PRIMARY ANTIBODY | α-enolase | β-enolase | γ-enolase | FIPV (N)¹ | FIPV (S)² | TGEV |
| FIPV positive serum from KSU | − | + | − | + | + | ND³ |
| case number 9190-2 | | | | | | |
| Feline infectious peritonitis Type | − | + | − | + | + | + |
| 1 (FIP-1) Antiserum-polyclonal | | | | | | |
| Feline infectious peritonitis Type | − | − | − | + | + | + |
| 2 (FIP-2) Antiserum-polyclonal | | | | | | |
| Mouse anti-feline coronavirus | − | − | − | + | − | − |
| FIPV Type 1 and 2 reactive | | | | | | |
| monoclonal | | | | | | |
| Human Non-Neuronal Enolase | + | − | − | + | + | − |
| (NNE)-rabbit polyclonal α-α- | | | | | | |
| enolase | | | | | | |
| Human C-terminus of α- | + | − | − | − | +/− | + |
| enolase/ENO1-goat polyclonal | | | | | | |
| Human N-terminus of α-enolase- | ++ | − | − | ++ | + | − |
| rabbit polyclonal | | | | | | |
| Human β-enolase/ENO3 | − | + | − | − | − | − |
| mouse IgG2a | | | | | | |
| Mouse monoclonal gamma | − | − | + | − | − | − |
| enolase antibody | | | | | | |
| Human γ-enolase/ENO2 | − | − | − | − | − | − |
| mouse IgGl | | | | | | |
| TGEV polyclonal serum | − | − | − | + | + | − |

¹FIPV (N) is FIPV nucleocapsid protein
²FIPV (S) is FIPV spike protein
³ND, not determined

The western blot analyses summarized in Table 1 show that antibodies which recognized the N-terminal domain of human alpha-enolase also recognized the nucleocapsid and spike proteins of FIPV. Antibodies that recognized the C-terminus domain of alpha-enolase reacted weakly with FIPV spike proteins, and did not react with FIPV nucleocapsid proteins. Antibodies that recognized either beta or gamma-enolase did not react with FIPV nucleocapsid or spike proteins. Therefore, the results suggest that the N-terminal domain of alpha-enolase shares immunogenic regions or domains with the spike and nucleocapsid proteins of FIPV. These results further suggest that vaccines which do not include antigens of FIPV that cross react with domains of alpha-enolase will be less likely to induce auto-antibodies in vaccinated animals, such as cats.

### Example 8

### Isolation of a Feline α-Enolase cDNA

Materials and Methods

Screening of Feline Uterus cDNA Library for Feline α-Enolase

For screening of feline α-enolase, feline uterus cDNA lambda uni-ZAP library (Stratagene, Cedar Creek, Tex., USA) was used. The cDNA library was screened by Southern blot. The cDNA library was combined with E. coli host strain, XL1-Blue MRF′, mixed NZY agarose, and then poured onto NZY agar plates. After incubating at 37° C. for 8 hrs, the plaques were transferred onto nylon membranes, denatured, neutralized, and fixed at 80° C. for 2 hrs. The human α-enolase cDNA (Open Biosystems, Huntsville, Ala., USA) was used as a probe. The human α-enolase cDNA was labeled with [α-³²P] CTP by Ready-To-Go™ DNA labeling beads (Amersham Biosciences Corp., Piscataway, N.J., USA). The hybridization was perform at 65° C. in hybridization solution (5×SET, 5× Denhardt's reagent, 1% SDS, 200 μg/ml denatured ssDNA, and 200 μg/ml heparin). The nylon membranes were washed for 30 mins twice with wash buffer I (2×SET and 5× Denhardt's reagent), for 30 mins four times with wash buffer II (2×SET and 0.5% SDS), and for 30 mins twice with wash buffer III (0.1×SET and 0.1% SDS). The membranes were exposed onto X-ray films, and then the films were developed. The positive plaques were rescued using the ExAssist® helper phage (Stratagene, Cedar Creek, Tex., USA) with E. coli host strain, SOLR™ (Stratagene, Cedar Creek, Tex., USA) by following manufacturer's instructions. The rescued clones were then sequenced.

RT-PCR for Amplifying 5′ Region of Feline α-Enolase cDNA

RNA was isolated from Crandall-Reese feline kidney (CRFK) cells with RNeasy® mini kit (Qiagen, Valencia, Calif., USA). RT-PCR was performed using Qiagen® onestep RT-PCR kit (Qiagen, Valencia, Calif., USA) with forward 5′-CACCATGTCTATTCTCAAGATCCA-3′ (SEQ ID NO: 11) and reverse 5′-CTTCTTTGTTCTCCAGGATGTTAG-3′ (SEQ ID NO: 12) primers. The reverse transcription reaction was performed at 50° C. for 30 minutes. The initial PCR was done at 95° C. for 15 minutes, and the standard PCR was done at 94° C. for 30 seconds, 48° C. for 30 seconds, 72° C. for 60 seconds, for 30 cycles and 72° C. for 30 minutes. The product was analyzed on a 1% agarose gel with ethidium bromide.

Cloning of the RT-PCR Product from RNA of CRFK Cells

The RT-PCR product was purified by Clontech NucleoTrap™ gel extraction kit (Clontech Laboratories, Inc., Palo Alto, Calif., USA). The purified cDNA was cloned with pGEM™-T easy vector system I (Promega Corporation, Madison, Wis., USA), and then was sequenced.

Results

Using a full-length human α-enolase cDNA (obtained from Open Clone Systems, MA) as the probe, three plaques were identified by their hybridization with the probe and the authenticity of the clones was confirmed by sequencing. The isolated full-length feline α-enolase cDNA has 1305 nucleotides (FIG. 7) and shares high homology with canine and orangutan α-enolase. Full-length feline α-enolase has been cloned in the pQE expression system and recombinant α-enolase was expressed in E. coli. The recombinant protein was purified by nickel chelation chromatography. The column purified protein can be used for antibody ELISA development.

### Example 9

### Dissociation of α-Enolase Antibodies from Circulating Immune Complexes by Acidification

In some embodiments, it may be necessary to treat the sample by acidification, with an optimum pH being 1.9, to dissociate α-enolase antibodies from Circulating Immune Complexes (CIC), for purpose of detection. For example, when samples were stored for more than three months, or in cats in middle to late stages of FIP (three months or longer after infection). Detection in post-mortem samples taken from the cerebral spinal fluid does not need acidification. Increasing level of free antibodies by acidification was done before. For example, in heartworm infections, the availability of the antigen is improved by acidification and detergent treatment of the serum (Steindl et al., 1998).

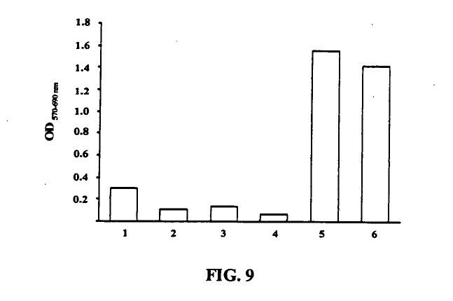

Serum and ascitic samples from cats that had died (n=10) were obtained. Diagnosis of those cats as having had FIP was confirmed by histopathology. The effect of acidification, length of treatment, and type of treatment on the release of enolase antibodies from CIC were studied. The amount of free α-enolase antibodies were measured by ELISA. Acidification at pH (2.0) for 30 seconds at 37° C. was sufficient to release enolase antibodies and dissociate the immune complex. Milder acidification (pH 3-7) did not help antibody extraction from the circulating immune complexes. See FIG. 9. Longer treatment and higher temperature treatments had detrimental effects on the antibody extraction (data not shown).

The data suggested that α-enolase antibodies occur in two phases during the course of FIP. During the initial phase of the disease, most of the antibody is soluble (“free”). Later, most of the antibody is present in CIC. This could be due to an increase in antibody affinity in later stages of FIP.

### Example 10

### α-Enolase Antibodies are Cytotoxic to Crandell Feline Kidney Cell Line Cells In Vitro

The sera from 30 cats exposed to FIP-CoV under field conditions were tested for cytotoxic activity using methylthiazoletetrazolium (MTT) assay. The assays were performed essentially as described (Denizot & Lang, J. Immunol. Methods (1986); Green et al., J. Immunol. Methods (1984)). Crandell Feline Kidney cells were used in the assays. The feline sera contain varying levels of α-enolase antibodies and cytotoxicity of the sera was found to positively correlate with levels of α-enolase antibodies in the sera.

### Example 11

### α-Enolase Antibodies are Specifically Found in FIPV-Infected Cats

Sera from clinical cases (of virus-infected cats) submitted to Kansas Veterinary Diagnostic Laboratory, Manhattan, Kans., were analyzed for the presence or absence of α-enolase antibodies. α-enolase antibodies were found in FIPV-infected cats. Like FIPV, Feline Immunodeficiency Virus (FIV) and Feline Leukemia Virus (FeLV) are also feline viruses that are associated with immune complex formation. However, no α-enolase antibodies were detected in sera known to be positive for FIV and FeLV.

What is claimed is:

1. A method for screening an individual of the Felidae family for FIP CoV exposure or infection, said method comprising:
a) determining whether or not a sample comprising circulating immune complexes from said individual comprises enolase.

2. The method of claim 1, wherein said determining comprises detection of said enolase, and wherein said detection is indicative that said individual has been exposed to a virulent form of FIP CoV.

3. The method of claim 1, wherein said enolase comprises the α-isoform and homo- or hetero-dimers of the α-isoform.

4. The method of claim 1, further comprising determining whether or not said sample comprises an antibody specific for enolase.

5. The method of claim 1, further comprising determining whether or not said sample comprises viral FIP RNA.

6. The method of claim 5, wherein said determining comprises detecting said viral FIP RNA using a polynucleotide probe specific for the 3′UTR of said viral FIP RNA.

7. The method of claim 1, wherein said sample comprises an antibody specific for enolase.

8. The method of claim 1, wherein said sample comprises viral FIP RNA.

9. The method of claim 1, wherein said determining comprises detecting said enolase using a technique selected from the group consisting of: a western blot, a northwestern blot, an ELISA, a lateral flow immunoassay, an immunohistochemistry technique, and a protein sequencing method.

10. The method of claim 1, wherein said sample is selected from the group consisting of serum, peritoneal fluid, thoracic fluid, cerebrospinal fluid, lymph, saliva, lachrymal fluid, aqueous or vitreous humor, ascites fluid, plasma, whole blood, a fresh biopsy sample, a fixed tissue sample, lavages, tracheal washings, and effusions of said individual.

11. A method for screening an individual of the Felidae family for FIP CoV exposure or infection, said method comprising:
a) determining whether or not a sample from said individual comprises an antibody specific for enolase.

12. The method of claim 11, wherein said sample comprises circulating immune complexes.

13. A method for determining whether or not a test vaccine for a multi-organ CoV is safe for administration comprising:
(a) administering said test vaccine to an individual;
(b) determining whether or not an elevated level of antibodies specific for enolase is produced in said individual relative to a control individual not administered said test vaccine, wherein an elevated level of antibodies specific for enolase is indicative that said test vaccine is not safe for administration.

14. The method of claim 13, wherein said multi-organ CoV is FIP, SARS, or a SARS-like virus.

15. An isolated antibody specific for Felidae enolase, wherein said isolated antibody is not specific for human enolase.

16. The isolated antibody of claim 15, wherein said antibody is derived from an individual of the Felidae family.

17. The isolated antibody of claim 15, wherein said antibody is a component of a circulating immune complex.

18. The isolated antibody of claim 17, wherein said Felidae enolase is selected from the group consisting of the alpha-enolase isoform, the gamma-enolase isoform, alpha-alpha enolase, gamma-gamma enolase, alpha-gamma enolase, and mixtures thereof.

19. An article of manufacture comprising an isolated antibody specific for Felidae enolase, wherein said isolated antibody is not specific for human enolase.

20. A method for evaluating if a test FIP vaccine has an increased tendency to induce an ADE response in a individual of the Felidae family, said method comprising:
(a) administering said test FIP vaccine to an individual of the Felidae family;
(b) determining whether or not, after said test FIP vaccine administration, said individual exhibits CICs comprising enolase in an elevated amount relative to a control individual not administered said test vaccine, wherein said elevated production of CICs is indicative that said test FIP vaccine has an increased tendency to induce an ADE response.

21. An isolated polynucleotide comprising a nucleic acid having 91% or higher sequence identity to SEQ ID NO:9.

22. The isolated polynucleotide of claim 21, wherein said nucleic acid is SEQ ID NO:9.

23. An isolated polynucleotide comprising a nucleic acid encoding a polypeptide having 97% or higher sequence identity to the amino acid sequence set forth in SEQ ID NO:10.

24. The isolated polynucleotide of claim 23, wherein said polypeptide is SEQ ID NO:10.

25. An isolated polypeptide comprising an amino acid sequence having 97% or higher sequence identity to SEQ ID NO:10.
